(19) United States
(12) Patent Application Publication
(10) Pub. No.: US 2004/0235753 A1
(43) Pub. Date: Nov. 25, 2004

(54) Oxytocin agonists

(76) Inventors: Gray Robert William Pitt, Tidworth Hampshire (GB); Michael Bryan Roe, Southampton (GB); David Philip Rooker, Southampton (GB)

Correspondence Address: Stephen A Bent, Foley & Lardner, Washington Harbour, 3000 K Street NW Suite 500, Washington, DC, 20007-5109, US

(21) Appl. No.: 10/482,102

(22) PCT Filed: Jun. 24, 2002
(86) PCT No.: PCT/GB02/02872

(30) Foreign Application Priority Data
Jun. 25, 2001 (GB) 0115515.9

(51) Int. Cl. A61K 38/04; C07K 5/04; C07D 417/02; C07D 413/02; C 07D 4 3/02

(52) U.S. Cl. 514/19; 514/210.2; 514/235.2; 514/254.01; 514/326; 514/423; 544/60; 544/141; 544/373; 546/208; 548/517; 548/532

(57) ABSTRACT

Compounds according to general formula 1, wherein G¹ is NR⁶R⁷ or a group according to general formula 3 and G² NR²⁴R²⁵ or a fused polycyclic group are novel. They are selective and potent oxytocin agonists. Pharmaceutical compositions of such compounds are useful in the treatment of, inter alia, erectile dysfunction.

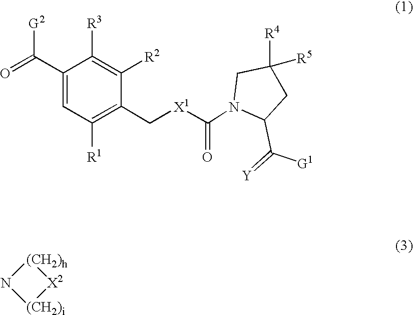

### FIELD OF THE INVENTION

The present invention relates to a series of non-peptide oxytocin agonists and to pharmaceutical compositions comprising such compounds. The compositions are useful for the treatment of certain physiological disorders, such as erectile dysfunction.

### BACKGROUND

### Neurophyseal Hormones

The neurophyseal hormones oxytocin (OT) and vasopressin (VP) are cyclic nonapeptides secreted by the posterior pituitary gland. The structure of oxytocin is shown below.

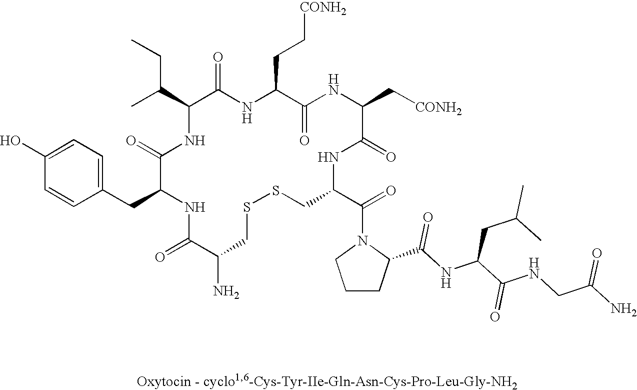

Vasopressin differs from oxytocin in that it has phenylalanine at position 3 in place of isoleucine and arginine at position 8 in place of leucine. Both hormones are synthesised in vivo as larger precursors, neurophysins, which are subject to post-translational processing to release the mature peptides. OT and VP act through a family of heptahelical receptors. Only one OT receptor has so far been well characterised, while three VP receptors are known. These are designated the V₁ₐ, V_{1b} and V₂ receptors.

The first target organs to be identified for OT were the uterus, where it is implicated in the onset and progress of labour, and mammary glands, where it is involved in the regulation of milk expression. Other organs also express OT receptors, and it is clear that OT has a range of physiological roles that have not been fully elaborated yet. In particular, it has been suggested that OT acting in the CNS is involved in the erectile response in males, and in the regulation of female sexual arousal. For example, OT is erectogenic when administered i.c.v. to male rats. It also has erectogenic activity when given i.v., but the doses required are up to two orders of magnitude greater, which is consistent with a central mode of action.

Vasopressin acts on the blood vessels, where it is a potent vasoconstrictor, and on the kidneys, where it promotes water reuptake leading to an antidiuretic effect.

### Oxytocin Agonists and Antagonists

A number of peptide analogues of OT are known in the literature. These include both agonists and antagonists. OT and its agonists are used, for example, to accelerate labour and to increase uterine muscle tone to control post-partum bleeding, and one antagonist, atosiban, has recently been registered as a treatment for pre-term labour. However, the peptidic nature of these compounds means that they are not likely to be bioavailable after oral dosing or to cross into the CNS. In order to get drugs that can be given orally and to be able to exploit the central effects of OT, attention has increasingly turned to non-peptides. As a result, there are many publications describing non-peptide OT antagonists in early-stage development. So far, however, there have been no reports of non-peptide OT agonists. This is not unexpected, as it is generally held that it is easier to find a receptor antagonist than an agonist.

So there remains a need for non-peptide OT receptor agonists. Such compounds should preferably be selective for the OT receptor over the VP receptors. They could be expected to show therapeutic utility in male and female sexual dysfunction, particularly male erectile dysfunction, in promoting labour, in controlling post-partum bleeding, in increasing milk let-down as well as a number of other indications.

### SUMMARY OF THE INVENTION

We describe herein a series of potent and specific OT receptor agonists. In a first aspect, the present invention comprises novel compounds according to general formula 1, and pharmaceutically acceptable salts thereof.

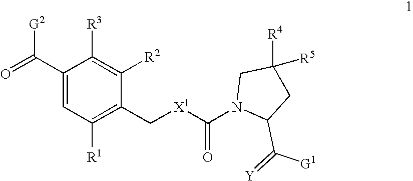

In general formula 1, G¹ is a group according to general formula 2 or 3.

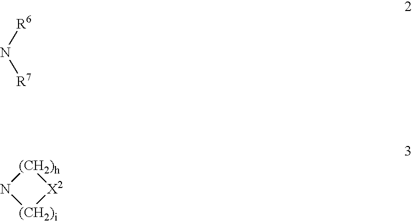

G² is a group according to general formula 4, 5, 6, 7, 8 or 9.

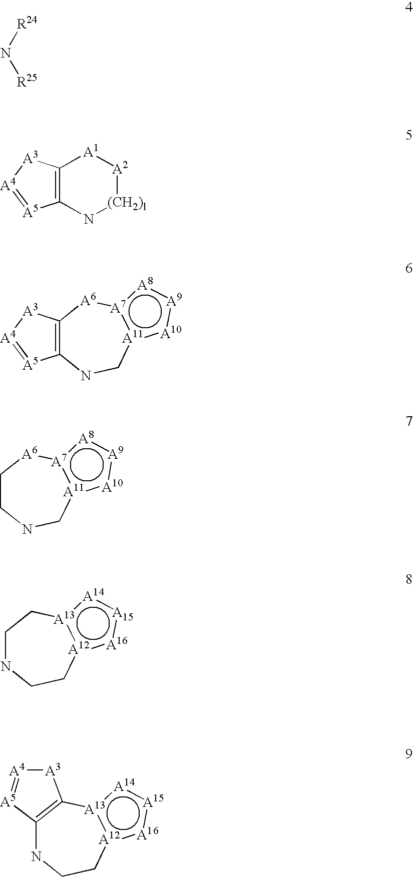

A¹ is CH₂, CH(OH), NH, N-alkyl, N-(CH₂)ₙ-R²⁷, O or S; A² is CH₂, CH(OH), C(═O) or NH;

A³ is S, NH, N-alkyl, —CH═CH— or —CH═N—; A⁴ and A⁵ are each CH or N; A⁶ is CH₂, NH, N-alkyl or O; A⁷ and A¹¹ are C or N; A⁸ and A⁹ are CH, N, NH, N(CH₂)ₘR²⁶ or S; A¹⁰ is —CH═CH—, CH, N, NH, N—(CH₂)ₘ-R²⁶ or S; A¹² and A¹³ are N or C and A¹⁴, A¹⁵ and A¹⁶ are NH, N—CH₃S, N or CH, provided that not more than one of A⁸, A⁹ and A¹⁰ is NH, N—(CH₂)ₘ-R²⁶ or S; that A⁷ and A¹¹ are not both simultaneously N; that neither A⁷ nor A¹¹ is N if one of A⁸, A⁹ is NH, N—(CH₂)ₘ-R²⁶ or S; that if A¹⁰ is —CH═CH— then A⁸ is N, A⁹ is CH and both A⁷ and A¹¹ are C; that if A¹⁰ is not —CH═CH— then one of A⁸, A⁹ and A¹⁰ is NH, N—(CH₂)ₘ-R²⁶ or one of A⁷ and A¹¹ is N; that not more than one of A¹⁴, A¹⁵ and A¹⁶ is NH, N—CH₃ or S; that A¹² and A¹³ are not both simultaneously N; that if one of A¹⁴, A¹⁵ and A¹⁶ is NH, N—CH₃ or S then A¹² and A¹³ are both C; and that one of A¹⁴, A¹⁵ and A¹⁶ is NH, N—CH₃ or S or one of A¹² and A¹³ is N.

X¹ is O or NH; X² is NR¹⁶, CH—NR¹⁷R¹⁸, CH—CH₂NR¹⁷R¹⁸, N⁺R¹⁹R²⁰, CH—N⁺R²¹R²²R²³ or CH—CH₂N⁺R²¹R²²R²³ and Y is O or S.

R¹, R² and R³ are each H, alkyl, O-alkyl, F, Cl or Br; R⁴ and R⁵ are each H, O-alkyl, O-benzyl or F, or R⁴ and R⁵ together are ═O, —O(CH₂)ₐO— or —S(CH₂)ₐS— and R⁶ is a group according to one of general formulae 10 to 25.

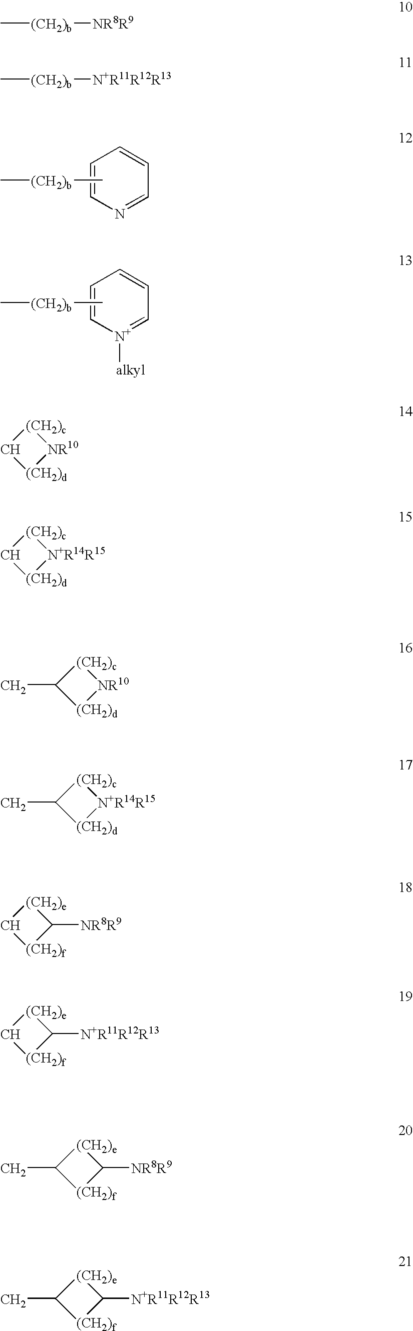

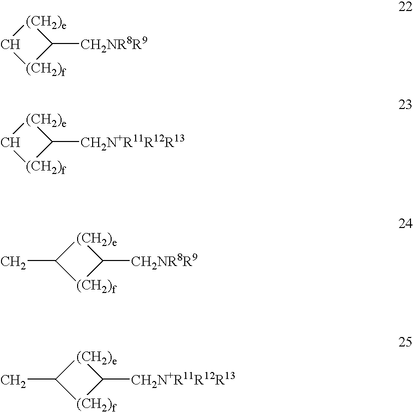

R⁷ is H, alkyl or any group as defined above for R³; R⁸, R⁹ and R¹⁰ are independently H or alkyl, or R⁸ and R⁹ together may be —(CH₂)_{g}-; R¹¹, R¹², R¹³, R¹⁴ and R¹⁵ are all alkyl, or R¹¹ and R¹² together or R¹⁴ and R¹⁵ together may be —(CH₂)_{g}-; R¹⁶, R¹⁷ and R¹⁸ are independently H or alkyl, or R¹⁷ and R¹⁸ together may be —(CH₂)ⱼ-; R¹⁹, R²⁰, R²¹, R²² and R²³ are all alkyl, or R¹⁹ and R²⁰ together or R²¹ and R²² together may be —(CH₂)ⱼ-; R²⁴ and R²⁵ are independently alkyl, Ar or —(CH₂)ₖ-Ar; R²⁶ is H, alkyl, optionally substituted phenyl, pyridyl, thienyl, OH, O-alkyl, NH₂, NH-alkyl, N(alkyl)₂, CO₂H, CO₂-alkyl, CONH₂, CONH-alkyl, CON(alkyl)₂, CN or CF₃, and R²⁷ is OH, O-alkyl, O-CO-alkyl, NH₂, NH-alkyl or N(alkyl)₂.

Ar is thienyl or optionally substituted phenyl.

a is 2 or 3, b is 1, 2 or 3; ; c is 1 or 2, d is 1, 2 or 3; e is 1 or 2; f is 1, 2 or 3; g is 4, 5 or 6; h is 1, 2 or 3; i is 1, 2, 3 or 4; j is 4, 5 or 6; k is 1, 2 or 3; l is 1 or 2; m is 1, 2 or 3, and n is 2, 3 or 4.

In a second aspect, the present invention comprises pharmaceutical compositions of these novel compounds, which compositions are useful for the treatment of, inter alia, male erectile dysfunction. In further aspects, the present invention comprises the use of such compositions in therapy and therapeutic methods using the compositions.

### DETAILED DESCRIPTION OF THE INVENTION

In a first aspect, the present invention comprises novel benzyl carbamates and ureas according to general formula 1.

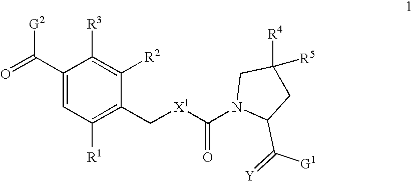

In this general formula the substituents R¹, R² and R³ are independently selected from hydrogen (H), alkyl groups, alkoxy (O-alkyl) groups, and the halogens fluorine (F), chlorine (Cl) and bromine (Br). Preferably, at least one of R¹, R² and R³ is H and at least one is not H. More preferably, one of R¹, R² and R³ is an alkyl group or a halogen and the others are H. Most preferably, R¹ is methyl or Cl and R² and R³ are both H.

The substituents R⁴ and R⁵ may be independently selected from H, O-alkyl, O-benzyl and F. Alternatively, R⁴ and R⁵ together may be ═O so as to form (together with the carbon atom to which they are attached) the carbonyl group of a ketone. Finally, R⁴ and R⁵ together may be —O(CH₂)ₐO— or —S(CH₂)ₐS—, where a is 2 or 3, so as to form (together with the carbon atom to which they are attached) a 1,3-dioxolane, a 1,3-dithiolane, a 1,3-dioxane or a 1,3-dithiane. Preferably, both R⁴ and R⁵ are H or one is H and the other is O-alkyl. More preferably, both R⁴ and R⁵ are H or one is H and the other is methoxy. Furthermore, in embodiments where one of R⁴ and R⁵ is F, it is preferred that the other should also be F.

The linking group X¹ is selected from oxygen (O) and unsubstituted nitrogen (NH). Preferably, X¹ is NH.

Y is selected from O and sulphur (S).

The group G¹ is a mono- or disubstituted nitrogen atom, such that the C(═Y)-G¹ bond is an amide or thoiamide bond. A requirement of G¹ is the presence of at least one basic nitrogen atom or a quaternary salt derived from such a basic nitrogen atom within the group. The basic nitrogen atom may be present as a primary, secondary or tertiary amino group or a pyridyl nitrogen. When G¹ includes more than one such basic nitrogen atom then these may be the same or different. Accordingly, G¹ is selected from an acyclic group according to general formula 2 and a cyclic group according to general formula 3.

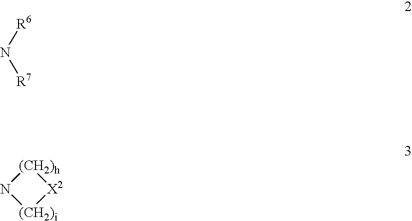

In general formula 2, R⁶ and R⁷ may be the same or different. R⁶ is selected from a group according to one of general formulae 10 to 25.

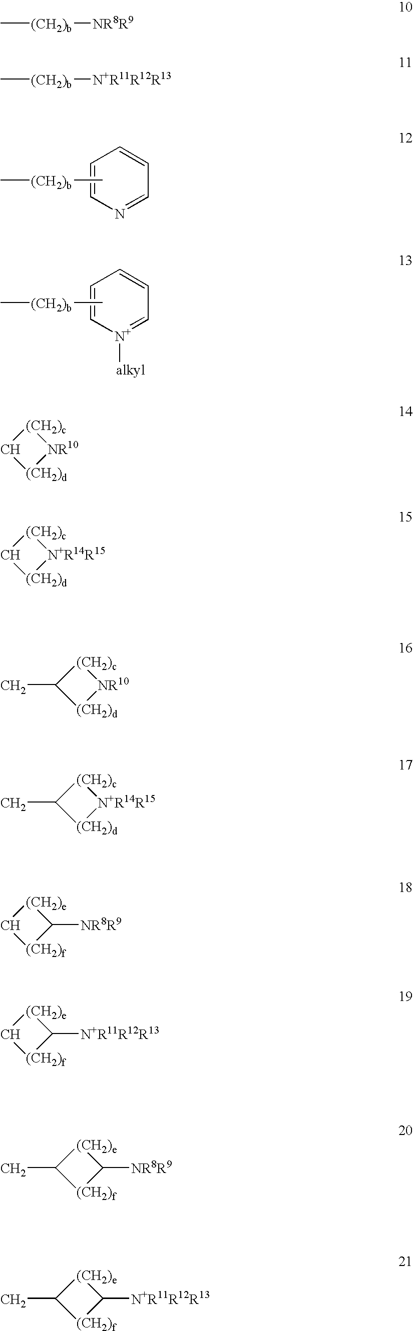

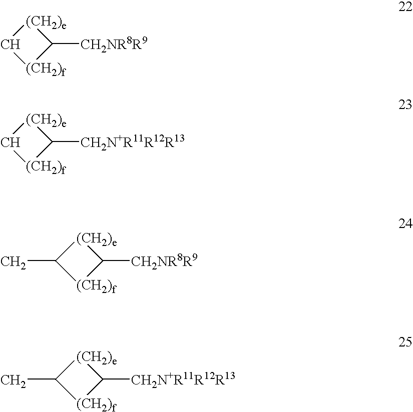

In the above it will be noted that, for each of the groups that includes a basic nitrogen atom (i.e. for general formulae 10, 12, 14, 16, 18, 20, 22 and 24) there is a corresponding quaternary derivative (i.e. 11, 13, 15, 17, 19, 21, 23 and 25 respectively). For the polymethylene spacer groups b is 1, 2 or 3; c is 1 or 2, d is 1, 2 or 3; e is 1 or 2 and f is 1, 2 or 3. R⁸, R⁹ and R¹⁰ are independently selected from H and alkyl. Alternatively R⁸ and R⁹ together may be a polymethylene chain —(CH₂)_{g}-, where g is 4, 5 or 6, so as to form, together with the nitrogen atom to which they are attached, a pyrrolidine, piperidine or perhydroazepine ring. R¹¹, R¹², R¹³, R¹⁴ and R¹⁵ are all alkyl, or R¹¹ and R¹² together or R¹⁴ and R¹⁵ together may be —(CH₂)_{g}-, again allowing for pyrrolidine, piperidine and perhydroazepine rings.

For the groups according to general formulae 12 and 13, the attachment to the pyridine ring may be at the 2- or 4-position.

R⁷ may be any of the foregoing groups described for R⁶, or it may be a non-basic group selected from H and alkyl.

In the group according to general formula 3, h is 1, 2 or 3 and i is 1, 2, 3 or 4. X² is selected from the basic groups NR¹⁶, CH—NR¹⁷R¹⁸ and CH—CH₂NR¹⁷R¹⁸ and the corresponding quaternary groups N⁺R¹⁹R²⁰, CH—N⁺R²¹R²²R²³ and CH—CH₂N⁺R²¹R²²R²³. R¹⁶, R¹⁷ and R¹⁸ are independently selected from H and alkyl, or R¹⁷ and R¹⁸ together may be —(CH₂)ⱼ-, where j is 4, 5 or 6, such that R¹⁷, R¹⁸ and the nitrogen atom to which they are attached may constitute a pyrrolidine, piperidine or perhydroazepine ring. R¹⁹, R²⁰, R²¹, R²² and R²³ are all alkyl, or R¹⁹ and R²⁰ together or R²¹ and R²² together may be —(CH₂)ⱼ-.

The group G² is a disubstituted nitrogen such that the C(═O)-G² bond is an amide bond. G² is selected from an acyclic group according to general formula 4, a fused bicyclic group according to general formulae 5, 7 and 8, and a fused tricyclic group according to general formulae 6 and 9.

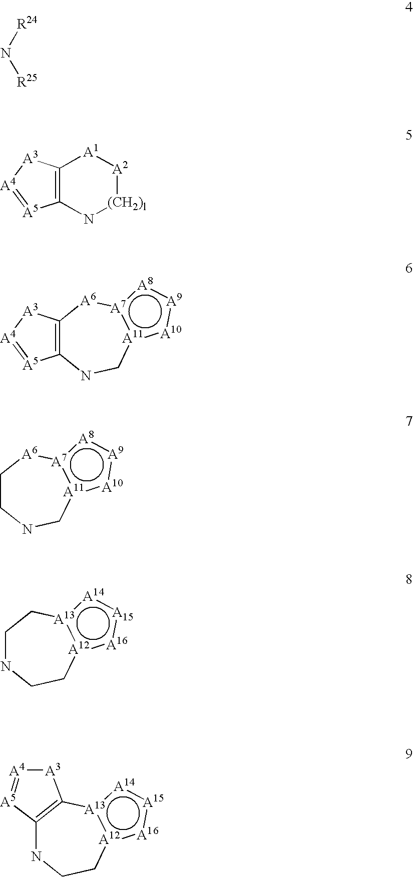

In general formula 4, R²⁴ and R²⁵ are independently selected from alkyl, Ar and —(CH₂)ₖ-Ar, where k is 1, 2 or 3 and Ar is selected from thienyl and optionally substituted phenyl. Suitable substituents for the phenyl group are alkyl groups, OH, alkoxy groups, halogen, NH₂, NH-alkyl and N(alkyl)₂. The phenyl group may be substituted with up to three such substituents which may be the same or different.

In general formula 5, A¹ is selected from CH₂, CH(OH), NH, N-alkyl, N—(CH₂)ₙ-R²⁷, O and S, where n is 2, 3 or 4 and R²⁷ is selected from OH, alkoxy groups, acyloxy (O—CO-alkyl) groups, NH₂, NH-alkyl and N(alkyl)₂. A² is selected from CH₂, CH(OH), C(═O) and NH, and I is 1 or more preferably 2. It is preferred that when A² is NH then A¹ is CH₂. It is also preferred that when A² is C(═O) then A¹ is NH or N-alkyl.

In general formulae 5, 6 and 9, A³ is selected from S, NH, N-alkyl, —CH═CH— and —CH═N— and A⁴ and A⁵ are each selected from CH and N. In a preferred embodiment, A³ is S and A⁴ and A⁵ are both CH, so as to form a thiophene ring. In another preferred embodiment, A³ is —CH═CH— and A⁴ and A⁵ are both CH, so as to form a benzene ring. In another preferred embodiment, A³ is —CH═N— and A⁴ and A⁵ are both CH, so as to form a pyridine ring. In another preferred embodiment, A³ is —CH═CH—, A⁴ is CH and A⁵ is N, again so as to form a pyridine ring.

In general formulae 6 and 7, A⁸ is selected from CH₂, NH, N-alkyl and O, A⁷ and A¹¹ are selected from C and N, A⁸ and A⁹ are selected from CH, N, NH, N—(CH₂)ₘ-R²⁶ and S and A¹⁰ is selected from —CH═CH—, CH, N, NH, N—(CH₂)ₘ-R²⁶ and S, where m is 1, 2 or 3 and R²⁶ is selected from H, alkyl, optionally substituted phenyl, pyridyl, thienyl, OH, O-alkyl, NH₂, NH-alkyl, N(alkyl)₂, CO₂H, CO₂-alkyl, CONH₂, CONH-alkyl, CON(alkyl)₂, CN and CF₃. Suitable substituents for the phenyl group are alkyl groups, OH, alkoxy groups, halogens, NH₂, NH-alkyl and N(alkyl)₂. The phenyl group may be substituted with up to three such substituents which may be the same or different.

The ring constituted by A⁷, A⁸, A⁹, A¹⁰ and A¹¹ is aromatic, and accordingly the groups must satisfy certain requirements. When A¹⁰ is —CH═CH— the ring is a six-membered ring. As such, it can only comprise atoms of the type —C(R)═ and —N═. Hence A⁷ and A¹¹ must both be C and A⁸ and A⁹ must be either CH or N. We have found that suitable activity is only obtained when A⁸ is N and A⁹ is CH. When A¹⁰ is not —CH═CH— then the ring is a five-membered ring. In this case one, and only one, of the atoms in the ring must be S or a trigonal nitrogen. In this context, a “trigonal nitrogen” is a nitrogen atom linked covalently to three different atoms. Two of these atoms are the immediate neighbours to the nitrogen atom in the five-membered ring. The third is a hydrogen, carbon or other atom linked to the five-membered ring. Thus it follows that, when A¹⁰ is not —CH═CH— then one (and only one) of A⁷, A⁸, A⁹, A¹⁰ and A¹¹ must be S or a trigonal nitrogen. Hence the selection of A⁷, A⁸, A⁹, A¹⁰ and A¹¹ is subject to the following restrictions.

1) If A¹⁰ is not —CH═CH— then one of A⁸, A⁹ and A¹⁰ is NH, N—(CH₂)ₘ-R²⁶ or S or one of A⁷ and A¹¹ is N.

2) Not more than one of A⁸, A⁹ and A¹⁰ may be NH, N—(CH₂)ₘ-R²⁶ or S.

3) A⁷ and A¹¹ may not both simultaneously be N.

4) Neither A⁷ nor A¹¹ may be N if one of A⁸, A⁹ and A¹⁰ is NH, N(CH₂)ₘR²⁶ or S.

In a preferred embodiment, A⁶ is NH. In another preferred embodiment, A⁸ is NH or N—(CH₂)ₘ-R²⁶. In a more preferred embodiment, A⁸ is NH or N—(CH₂)ₘ-R²⁶, A⁹ is N and A¹⁰ is CH.

In general formulae 8 and 9, A¹² and A¹³ are selected from N and C and A¹⁴, A¹⁵ and A¹⁶ are selected from NH, N—CH₃, S, N and CH. Again, these atoms constitute an aromatic five-membered ring and so there must be one, and only one, S or trigonal nitrogen. Hence the selection of A¹², A¹³, A¹⁴, A¹⁵ and A¹⁶ is subject to the following restrictions.

1) One of A¹⁴, A¹⁵ and A¹⁶ is NH, N—CH₃ or S or one of A¹² and A¹³ is N.

2) Not more than one of A¹⁴, A¹⁵ and A¹⁶ is NH, N—CH₃ or S.

3) A¹² and A¹³ may not both simultaneously be N.

4) If one of A¹⁴, A¹⁵ and A¹⁶ is NH, N—CH₃ or S then A¹² and A¹³ are both C

As used herein, the term “alkyl” is intended to designate lower alkyl groups, i.e. saturated hydrocarbon groups of between one and six carbon atoms, including linear, branched and cyclic alkyl groups. Examples of “alkyl” include, but are not limited to; C₁-methyl, C₂-ethyl, C₃-propyl, isopropyl, cyclopropyl, C₄-n-butyl, sec-butyl, isobutyl, tert-butyl, cyclobutyl, cyclopropylmethyl, methylcyclopropyl, C₅-n-pentyl, neopentyl, cyclopentyl, cyclopropylethyl, dimethylcyclopropyl, and C₆-n-hexyl, cyclohexyl, bicyclo[3.1.0]hexyl.

Those compounds according to the present invention that contain a basic nitrogen atom are capable of forming addition salts with protic acids such as hydrochloric acid, sulphuric acid, phosphoric acid, acetic acid, trifluoroacetic acid, benzoic acid, maleic acid, citric acid, fumaric acid, methanesulphonic acid and the like. Those compounds that contain a quaternary nitrogen atom will exist as salts with an appropriate counterion such as chloride, bromide, iodide, sulphate, phosphate, acetate, trifluoroacetate, benzoate, maleate, citrate, fumarate, methanesulphonate and the like. The compounds of the present invention may also contain an acidic group, such as a carboxylic acid group at R²⁶. These compounds may exist as inner salts (zwitterions) or as salts such as sodium, potassium, magnesium, calcium or tetra-alkylammonium salts. To the extent that such salts are pharmaceutically acceptable, they are included within the scope of the present invention.

The compounds according to the present invention all have at least one stereogenic centre (“asymmetric carbon atom”) and so may exhibit optical isomerism. The scope of the present invention includes all epimers, enantiomers and diastereomers of compounds according to general formula 1, including single isomers, mixtures and racemates.

Particularly preferred embodiments within the present invention are those compounds that combine two or more of the preferred features described above. One such particularly preferred embodiment is a thioamide according to general formula 26.

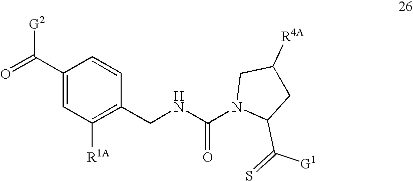

In general formula 26, R^{1A} is methyl or Cl and R^{4A} is H or O-methyl. G¹ and G² are as previously defined.

Another particularly preferred embodiment is an amide according to general formula 27.

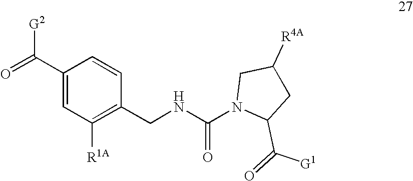

In general formula 27, R^{1A}, R^{4A}, G¹ and G² are as previously defined.

Another particularly preferred embodiment is a compound according to general formula 28.

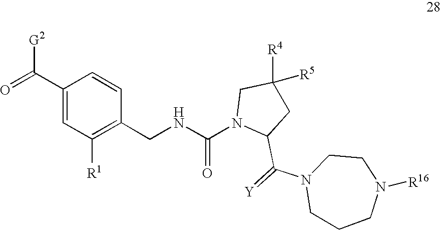

In general formula 28, R¹, R⁴, R⁵, R¹⁶, G² and Y are as previously defined.

Another particularly preferred embodiment is a compound according to general formula 29.

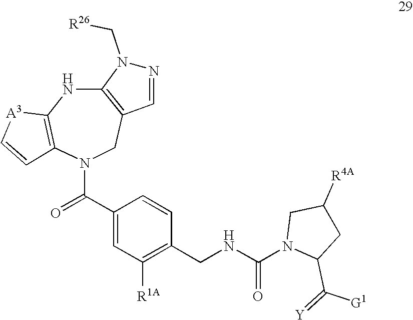

In general formula 29, R^{1A}, R^{4A}, R²⁶, A³, G¹ and Y are as previously defined.

A most preferred embodiment is a compound according to general formula 30.

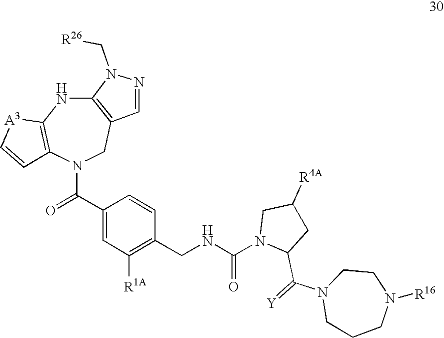

In general formula 30, R^{1A}, R^{4A}, R¹⁶, R²⁶, A³ and Y are as previously defined.

Individual preferred compounds within the invention include:

4-methyl-1-(N-(2-methyl-4-(2,3,4,5-tetrahydro-1,5-benzodiazepin4-on-1-ylcarbonyl)-benzylcarbamoyl)-L-thioprolyl)perhydro-1,4-diazepine,

4-methyl-1-(N-(2-methyl-4-(1-methyl-4,10-dihydropyrazolo[5,4-b][1,5]benzodiazepin-5-yl-carbonyl)benzylcarbamoyl)-L-thioprolyl)perhydro-1,4-diazepine,

4,4-dimethyl-1-(N-(2-methyl-4-(1-methyl-4,10-dihydropyrazolo[5,4-b][1,5]benzodiazepin-5-ylcarbonyl)benzylcarbamoyl)-L-thioprolyl)perhydro-1,4-diazepinium iodide,

4-methyl-1-(N-(2-methyl-4-(5,6,7,8-tetrahydrothieno[3,2-b]azepin-4-ylcarbonyl)benzylcarbamoyl)-L-thioprolyl)perhydro-1,4-diazepine,

4-methyl-1-(N-(2-methyl-4-(5,6,7,8-tetrahydrothieno[3,2-b]azepin-4-ylcarbonyl)benzyloxycarbonyl)-L-prolyl)perhydro-1,4-diazepine,

(4R)-N^{α}-(2-chloro-4-(5,6,7,8-tetrahydrothieno[3,2-b]azepin-4-ylcarbonyl)benzylcarbamoyl)-4-methoxy-L-proline-N-methyl-N-(2-picolyl)amide, and

1-((4R)-N^{α}-(2-chloro-4-(5,6,7,8-tetrahydrothieno[3,2-b]azepin-4-ylcarbonyl)benzylcarbamoyl)-4-methoxy-L-prolyl)-4-(1-pyrrolidinyl)piperidine

The compounds of the present invention can be prepared by standard chemical manipulations. In general, compounds according to general formula 1 can be considered to consist of four component parts:

Component C¹ corresponding to G²

Component C² corresponding to the substituted benzoyl unit

Component C³ corresponding to the pyrrolidine unit

Component C⁴ corresponding to G¹.

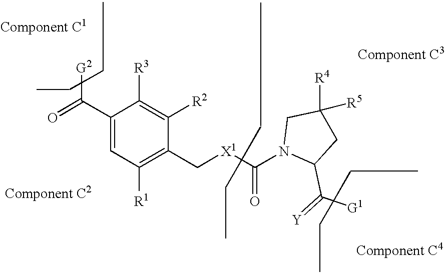

Intermediates corresponding to these components are prepared and then assembled to give the final product. These four components are:

| | | |
|---|---|---|
| (i) | for C¹, a secondary amine | G²—H |
| (ii) | for C², a substituted benzoic acid | 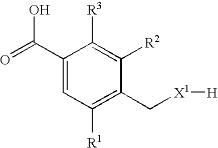 |
| (iii) | for C³, a proline derivative | 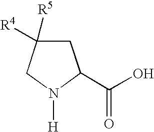 |
| (iv) | for C⁴, a primary or secondary | H—G¹ |
| | amine | |

It will be recognised that the substituted benzoic acid for C² and the proline derivative for C³ both have two functional groups that will need temporary protection during the assembly of the final compound. The principles of functional group protection are well known in the art and are described in, for example, J. F. W. McOmie, “Protective Groups in Organic Chemistry”, Plenum Press, 1973; T. W. Greene and P. G. M. Wuts, “Protective Groups in Organic Synthesis”, 2^{nd} edition, John Wiley, 1991; and P. J. Kocienski, “Protecting groups”, Georg Thieme Verlag, 1994. The carboxylic acid group will usually be protected as an ester, such as the methyl, benzyl or tert-butyl ester. The secondary amine of the proline and the primary amine of the benzoic acid (when X¹═NH) will usually be protected as a carbamate derivative such as the tert-butyl carbamate (BOC derivative), the benzyl carbamate (CBZ or more simply Z derivative) or the 9-fluorenylmethyl carbamate (Fmoc derivative). When X¹═O the resulting alcohol function will usually be protected as an ester such as an acetate, or an ether such as a methoxymethyl, tetrahydropyranyl or trialkylsilyl ether. Other functional groups may require protection. For example, the group G¹ may include one or more primary or secondary amino groups which may need protection. In the following general description of the synthetic methodology it will be assumed that such protection is used when necessary.

(i) Preparation of Secondary Amine for C¹

Acyclic secondary amines corresponding to HNR²⁴R²⁵ are well known. Many are items of commerce. Those that are not may be prepared according to published methods or by simple modification of such methods. Some particularly useful methods are listed below.

a) Alkylation

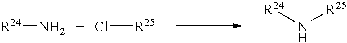

(This method is only applicable in cases where further alkylation can be avoided.)

b) Reductive Amination

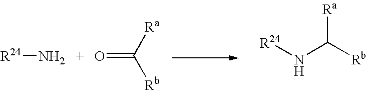

(where R^{a}CHR^{b} corresponds to R²⁵)

c) Amide Reduction

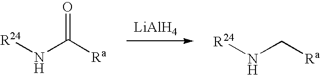

(where R^{a}CH₂ corresponds to R²⁵)

The starting amide can itself be prepared using well known methods.

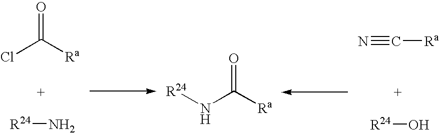

Secondary amines corresponding to C¹ where G² is a group according to general formulae 5-9 are generally not commercially available. They can be prepared according to published methods, or by obvious modifications of such methods. Particularly useful methods are described in: Aranapakam et al., Bioorg. Med. Chem. Lett. 1993, 1733; Artico et al., Farmaco. Ed. Sci. 24, 1969, 276; Artico et al., Farmaco. Ed. Sci. 32, 1977, 339; Chakrabarti et al., J. Med. Chem. 23, 1980, 878; Chakrabarti et al., J. Med. Chem. 23, 1980, 884; Chakrabarti et al., J. Med. Chem. 32, 1989, 2573; Chimirri et al., Heterocycles 36, 1993, 601; Grunewald et al., J. Med. Chem. 39, 1996, 3539; Klunder et al, J. Med. Chem. 35, 1992, 1887; Liegéois et al., J. Med. Chem. 37, 1994, 519; Olagbemiro et a., J. Het. Chem. 19, 1982, 1501; Wright et al, J. Med. Chem. 23, 1980, 462; Yamamoto et al., Tet. Lett. 24, 1983, 4711; and International patent application, publication number WO99/06403.

(ii) Preparation of Substituted Benzoic Acid for C²

Substituted benzoic acids corresponding to C² are not generally items of commerce, but they can be prepared using published methods or obvious variations of such methods. The main challenge is generally the elaboration of the —CH₂X¹H functionality at the 4-position. Some useful transformations are listed below.

a) Bromination/Substitution

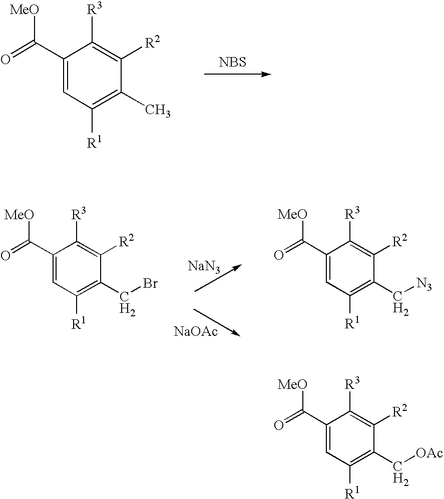

b) Sandmeyer Reaction/Reduction

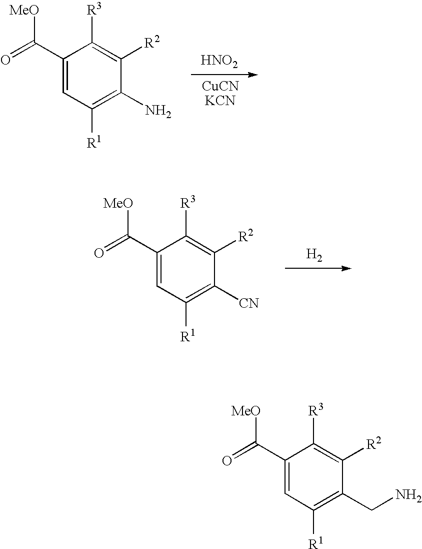

(iii) Preparation of Proline Derivative for C³

Proline and hydroxyproline derivatives are items of commerce. Other proline derivatives corresponding to C³ can be prepared according to the methods set out in Dugave et al., Tet. Lett. 39, 1998, 1169; Petrillo et al., J. Med. Chem. 31, 1988, 1148; and Smith et al., J. Med. Chem. 31, 1988, 875.

(iv) Preparation of Primary or Secondary Amine for C⁴

As noted in (i) above, many amines are commercially available and others are readily accessible using well established chemistry. The quaternary ammonium salts can generally be prepared by treating a primary, secondary or tertiary amine with an excess of alkylating agent.

With the four components, suitably protected if necessary, in hand, the assembly of the final compound requires the formation of three bonds: between C¹ and C², between C² and C³, and between C³ and C⁴. These bond-forming steps may be taken in any order. Thus, the following sequences can be proposed:

C¹+C²→C¹C²→C¹C²C³→C¹C²C³C⁴

C³+C⁴→C³C⁴→C²C³C⁴→C¹C²C³C⁴

C²+C³→C²C³→C²C³C⁴→C¹C²C³C⁴

C²+C³→C²C³→C¹C²C³→C¹C²C³C⁴

C¹+C²→C¹C²; C³+C⁴→C³C⁴; C¹C²+C³C⁴→C¹C²C³C⁴

(i) Formation of C¹-C² Bond

The bond between C¹ and C² is a simple amide bond. The chemistry for making such bonds from a carboxylic acid and a secondary amine is well known in the art of organic synthesis, and particularly in the field of peptide synthesis. The carboxylic acid may be converted into a more reactive species such as an acid chloride (using, for example oxalyl chloride or thionyl chloride) or a mixed anhydride (using isobutyl chloroformate). This reactive species is then added to the secondary amine in a suitable solvent, generally an aprotic solvent such as dichloromethane or dimethylformamide, in the presence of a base such as triethylamine or 4-dimethylaminopyridine, and the reaction is allowed to proceed at a temperature between −20° C. and the boiling point of the solvent. The choice of temperature and the time allowed for the reaction will depend on the reactivity of the two components.

Alternatively, the carboxylic acid and the secondary amine may be mixed in a suitable solvent as above, optionally in the presence of a base, and a condensing agent added. Suitable condensing agents include carbodiimides, such as dicyclohexylcarbodiimide (DCC) and N-ethyl-N′-dimethylaminopropylcarbodiimide (EDC, also WSCDI for water-soluble carbodiimide), phosphorus reagents such as (benzotriazol-1-yloxy)-tris(dimethylamino)phosphonium hexafluorophosphate (BOP), (benzotriazol-1-yloxy)-tripyrrolidinophosphonium hexafluorophosphate (PyBOP®) and bromotripyrrolidinophosphonium hexafluorophosphate (PyBroP®), and ureas such as O-(benzotriazol-1-yl)-N,N,N′,N′-tetramethyluronium hexafluorophosphate (HBTU).

(ii) Formation of C²-C³ Bond

The bond between C² and C³ is a carbamate (when X¹═O) or a urea (when X¹═NH). The first step in the formation of this bond is generally to react the proline derivative with phosgene or a phosgene equivalent such as trichloromethyl chloroformate, bis(trichloromethyl)carbonate or carbonyldiimidazole. Again, an aprotic solvent and a tertiary amine base will generally be used. The intermediate formed in this step is usually not isolated. The alcohol (X¹═O) or amine (X¹═NH) is added and the reaction is allowed to continue, directly forming the carbamate or urea. As an alternative, when X¹═NH the reactive intermediate may be formed by the reaction of C² with the phosgene equivalent and the proline added in the second part of the synthesis.

(iii) Formation of C³-C⁴ Bond

When Y═O, the bond between C³ and C² is a simple amide bond, and it may be formed by the methods described above for the formation of the C¹-C² bond. When Y═S, the bond is a thioamide. This is generally prepared by first making the simple amide and then treating this with Lawesson's reagent (2,4-bis(4-methoxyphenyl)-1,3-dithia-2,4-diphosphetane-2,4-disulfide).

The compounds according to the present invention are useful in human and animal therapy. When so used, they will generally be formulated in an appropriate manner. Thus a second aspect of the present invention is a pharmaceutical formulation that includes a compound as described above as an active ingredient. A third aspect of the present invention is the use of a compound according to the first aspect in the manufacture of such a composition.

The composition according to the present invention may be presented in any form that is known in the art. For example, the formulation may be presented as a tablet, capsule, powder, suppository, cream, solution or suspension, or in a more complex form such as an adhesive patch. The formulation will generally include one or more excipients, such as diluents, bulking agents, binding agents, dispersants, solvents, preservatives, flavoring agents and the like. Where the formulation is presented as a tablet or capsule the excipients may optionally include one or more agents to control the release of the active species, such as a coating of a polymer that is insoluble at low pH but soluble at neutral or high pH. Such a coating (known as an “enteric coating”) prevents the release of the active agent in the stomach but allows its release in the intestines. The formulation may also include one or more additional pharmacologically active species. Preferably the formulation includes no such additional active agents.

In further aspects, the present invention comprises the use of such compositions, and hence of the compounds of the invention, in human and animal therapy, and methods of treatment involving such use of the compositions and compounds. The compounds of the present invention are potent and selective oxytocin receptor agonists, and so the compositions are useful in the treatment of conditions for which inadequate oxytocin-like activity is implicated in the pathophysiology. Such conditions include, but are not limited to: sexual disorders such as male erectile dysfunction and ejaculatory disorders, female sexual dysfunction, cancer of the prostate, breast, ovary and bones, osteoporosis, benign prostatic hyperplasia, post-partum bleeding, and depression. The compositions may also be used to induce labour or delivery of the placenta, to decrease arterial blood pressure, to decrease exaggerated responses to stress and to increase the nociceptive threshold.

In a preferred embodiment, the composition is used to treat male or female sexual dysfunction, and more preferably erectile dysfunction.

When used as therapeutic agents, the compositions of the present invention may be administered by any appropriate route that is known in the art. For example, they may be administered by the oral, buccal, sublingual, rectal, intravaginal, nasal, pulmonary or transdermal routes. Alternatively, they may be given by injection, including intravenous, subcutaneous and intramuscular injection. The amount given will be determined by the attending physician taking into consideration all appropriate factors. Generally a single dose will comprise between 0.1 mg and 1000 mg, preferably between 1 mg and 250 mg, of active compound. The dose may be given on a single occasion or repeatedly. When given repeatedly, it may be given at regular intervals, such as once, twice or three times daily, or on demand, according to the condition being treated.

For long-term treatment an alternative to repeated dosing may be the administration of a depot dose. For this method of administration the active agent is generally introduced into a matrix of biodegradable polymer, such as a copolymer of lactic and glycolic acids, and the formulation is given either s.c. or i.m. so as to form a deposit from which the active agent is released as the polymer degrades.

The foregoing description is further illustrated in the following examples, which are intended to demonstrate the application of the invention but not to limit the scope thereof.

### EXAMPLES

The following abbreviations have been used:

Bu butyl—alkyl residues may be further denoted as n (normal, i.e. unbranched), i (iso) and t (tertiary)

DIEA N,N-diisopropylethylamine

DMF dimethylformamide

Et ethyl

HOBt 1-hydroxybenzotriazole

HPLC high pressure liquid chromatography

hr hour(s)

Me methyl

MS mass spectrum

NMR nuclear magnetic resonance spectrum

OVA ornithine vasotocin analogue

pet. ether petroleum ether boiling in the range 60-80° C.

Ph phenyl

Pn pentyl

Pr propyl

RT room temperature

THF tetrahydrofuran

WSCD water-soluble carbodiimide (N-ethyl-N′-(3-dimethylaminopropyl)carbodiimide hydrochloride

Examples 1-10 describe the synthesis of intermediates. Compounds according to the present invention are described in Examples 11 to 105.

### EXAMPLE 1

### 1-Benzyl-4,10-dihydropyrazolo[5,4-b][1,5]benzodiazepine

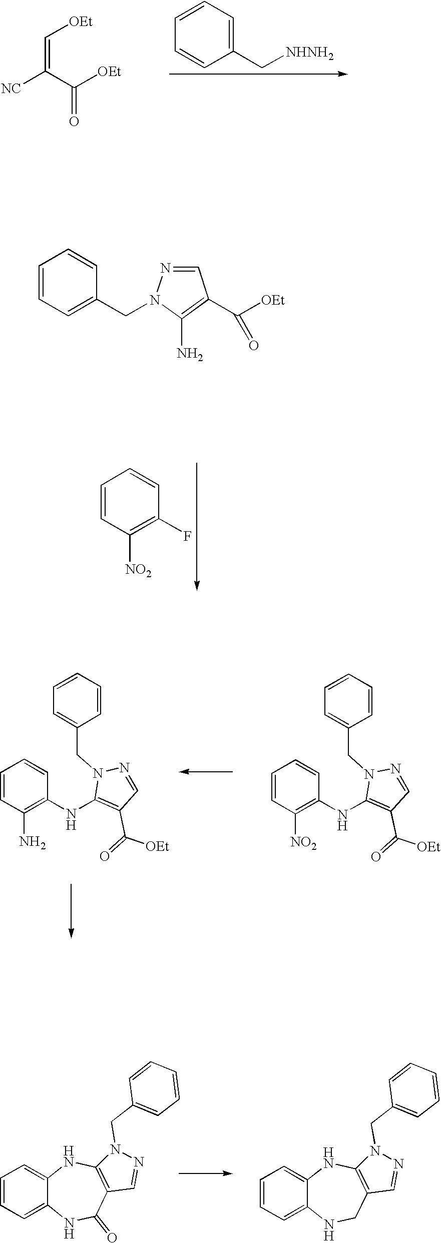

### 1A: Ethyl 5-amino-1-benzylpyrazole-4-carboxylate

Benzylhydrazine dihydrochloride (4.29 g, 22 mmol) was added to a solution of ethyl (ethoxymethylene)cyanoacetate (3.38 g, 20 mmol) and triethylamine (6.15 ml, 44 mmol, 2eq) in ethanol (40 ml) and the mixture was heated at reflux for 18 hr. The solvent was removed in vacuo and the residue was purified by flash chromatography on silica gel (eluant 60% pet. ether/40% ethyl acetate) to yield a pale yellow solid identified as ethyl 5-amino-1-benzylpyrazole-4-carboxylate (4.3 g, 88%).

### 1B: Ethyl 1-benzyl-5-(2′-nitrophenylamino)pyrazole-4-carboxylate

Sodium hydride (60% dispersion in oil, 520 mg, 13 mmol) was added portionwise to a suspension of ethyl 5-amino-1-benzylpyrazole-4-carboxylate (2.2 g, 9 mmol) in anhydrous THF (30 ml) at 0° C. The mixture was allowed to warm to room temperature and stirred for 2 hr then 1-fluoro-2-nitrobenzene (1.26 g, 9 mmol) was added and the resultant deep purple suspension was stirred at RT for 18 hr. 1M KHSO₄ was added to quench the reaction and the solvent was removed in vacuo. The residue was dissolved in ethyl acetate and the solution was washed with 0.3M KHSO₄, sat. NaHCO₃ and brine, dried over Na₂SO₄ and concentrated in vacuo. The residue was purified by flash chromatography on silica gel (eluant 75% pet. ether/25% ethyl acetate) to yield ethyl 1-benzyl-5-(2′-nitrophenylamino)pyrazole-4-carboxylate (2.5 g, 76%).

MS [M+H]⁺366.8

### 1C: Ethyl 5-(2′-aminophenylamino)-1-benzylpyrazole-4-carboxylate

Ethyl 1-benzyl-5-(2′-nitrophenylamino)pyrazole-4-carboxylate (2.5 g, 6.8 mmol) was dissolved in ethyl acetate/ethanol (1:1, 100 ml) and hydrogenated over 10% Pd/C catalyst for 70 minutes. The mixture was filtered through Celite® filter agent and the filtrate was concentrated in vacuo to give a white solid identified as ethyl 5-(2′-aminophenylamino)-1-benzylpyrazole-4-carboxylate (1.5 g, 86%).

MS [M+H]⁺337.2

### 1D: 1-Benzyl-4,10-dihydropyrazolo[5,4-b][1,5]benzodiazepin-4(5H)-one

A solution of ethyl 5-(2′-aminophenylamino)-1-benzylpyrazole-4-carboxylate (1.75 g, 5.2 mmol) in acetic acid/2-propanol (1:9, 40 ml) was heated at reflux for 3 days. The solvent was removed in vacuo and the residue was azeotroped with toluene to give an off-white solid that was purified by flash chromatography on silica gel (eluant 35% pet. ether/65% ethyl acetate) to yield a white solid identified as 1-benzyl-4,10-dihydro-pyrazolo[5,4-b][1,5]benzodiazepin-4(5H)-one (780 mg, 52%).

MS [M+H]⁺291.1

### 1E: 1-Benzyl-4,10-dihydropyrazolo[5,4-b][1,5]benzodiazepine

LiAlH₄ (365 mg, 10 mmol) was added portionwise to a suspension of 1-benzyl-4,10-dihydropyrazolo[5,4-b][1,5]benzodiazepin-4(5H)-one (780 mg, 2.7 mmol) in anhydrous THF (15 ml) at 0° C. over 10 min. The resulting suspension was heated at reflux for 18 hr, then allowed to cool to room temperature. A further portion of LiAlH₄ (90 mg, 2.5 mmol) was added and the mixture was heated at refluxed for 3 hr. The mixture was cooled to 0° C., 35% ammonia solution (1 ml) was added dropwise over 10 min and the mixture was stirred at RT for 1 hr. The resulting suspension was filtered through Celite® filter agent and the filtrate was concentrated in vacuo to give a white solid identified as 1-benzyl-4,10-dihydropyrazolo[5,4-b][1,5]benzodiazepine (450 mg, 60%).

MS [M+H]⁺276.9

### EXAMPLE 2

### 1-Methyl-4,10-dihydropyrazolo[4,5-c]pyrido[2,3-b][1,4]diazepine

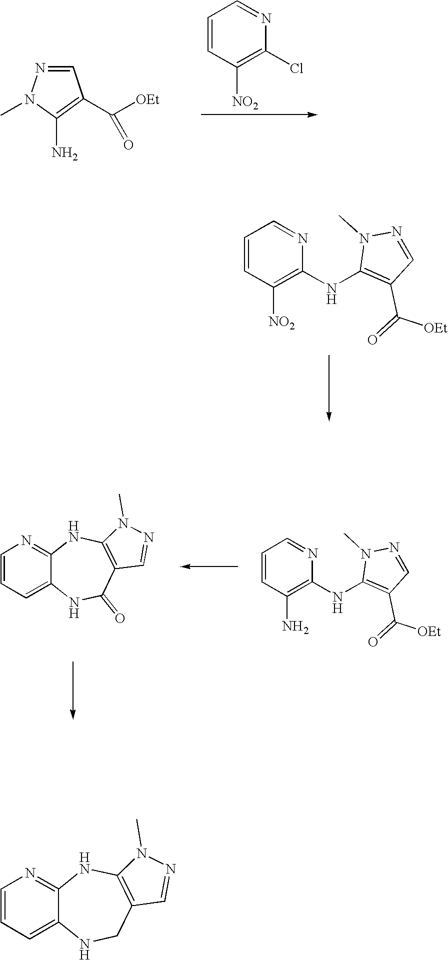

### 2A: Ethyl 1-methyl-2-(3′-nitro-2′-pyridylamino) pyrazole-4-carboxylate

Sodium hydride (60% dispersion in oil, 600 mg, 15 mmol) was added portionwise to a suspension of ethyl 5-amino-1-methylpyrazole-4-carboxylate (1.69 g, 10 mmol) in anhydrous THF (15 ml) at 0° C. The mixture was stirred for 2 hr at RT then 2-chloro-3-nitropyridine (1.58 g, 10 mmol) was added and the resulting deep red suspension was stirred at RT for 18 hr. 1 M KHSO₄ was added to quench the reaction and the solvent was removed in vacuo. The residue was dissolved in ethyl acetate and the solution was washed with 0.3M KHSO₄, sat. NaHCO₃ and brine, dried over Na₂SO₄ and concentrated in vacuo. The residue was purified by flash chromatography on silica gel (eluant 30% pet. ether/70% ethyl acetate) to give ethyl 1-methyl-2-(3′-nitro-2′-pyridylamino)pyrazole-4-carboxylate (1.95 g, 67%).

MS [M+H]⁺292.0

### 2B: Ethyl 2-(3′-amino-2′-pyridylamino)-1-methylpyrazole-4-carboxylate

A solution of ethyl 1-methyl-2-(3′-nitro-2¹-pyridylamino)pyrazole-4-carboxylate (1.95 g, 6.7 mmol) in ethanol (100 ml) was hydrogenated over 10% Pd/C catalyst for 3 hr. The reaction mixture was filtered through Celite® filter agent and the filtrate was concentrated in vacuo to give a white solid identified as ethyl 2-(3′-amino-2′-pyridylamino)-1-methyl-pyrazole-4-carboxylate (1.5 g, 86%).

### 2C: 1-Methyl-4,10-dihydropyrazolo[4,5-c]pyrido[2,3-b][1,4]diazepin-4(5H)-one

A solution of ethyl 2-(3′-amino-2′-pyridylamino)-1-methylpyrazole-4-carboxylate (1.5 g, 5.75 mmol) in acetic acid/2-propanol (1:9, 50 ml) was heated at reflux for 3 days. The solvent was removed in vacuo and the residue was azeotroped with toluene. The residue was purified by recrystallization from ethanol and then flash chromatography on silica gel (eluant 95% chloroform/4% methanol/1% acetic acid) to give a white solid identified as 1-methyl-4,10-dihydropyrazolo[4,5-c]pyrido[2,3-b][1,4]diazepin-4(5H)-one (560 mg, 45%).

### 2D: 1-Methyl-4,10-dihydropyrazolo[4,5-c]pyrido[2,3-b][1,4]diazepine

LiAlH4 (365 mg, 10 mmol) was added portionwise to a suspension of 1-methyl-4,10-dihydropyrazolo[4,5-c]pyrido[2,3-b][1,4]diazepin-4(5H)-one (560 mg, 2.6 mmol) in anhydrous THF (30 ml) at 0° C. over 10 minutes. The resulting suspension was heated at reflux for 18 hr. The reaction was cooled to 0° C. and 35% ammonia solution (1 ml) was added dropwise over 10 minutes, then the mixture was stirred at RT for 1 hr. The resulting suspension was filtered through Celite® filter agent and the filtrate was concentrated in vacuo to give a white solid identified as 1-methyl-4,10-dihydro-pyrazolo[4,5-c]pyrido[2,3-b][1,4]diazepine (410 mg, 78%).

MS [M+H]⁺202.1.

### EXAMPLE 3

### tert-Butyl N-methyl-N-(2-picolyl)carbamate

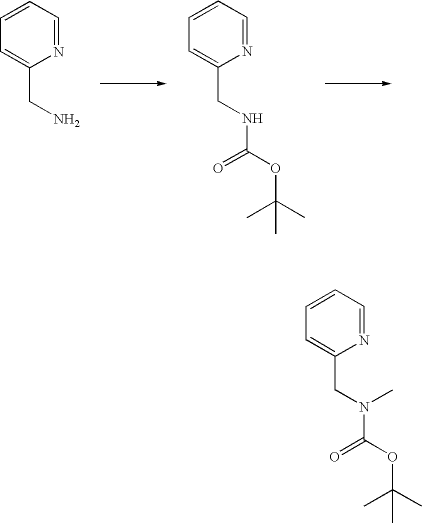

Di-tert-butyl dicarbonate (546 mg, 2.5 mmol) was added to an ice-cold solution of 2-(aminomethyl)pyridine (260 mg, 2.4 mmol) and triethylamine (340 μl, 2.4 mmol) in dichloromethane (10 ml). The solution was stirred at RT for 2 hr and the solvent was removed in vacuo. The residue was dissolved in anhydrous THF (10 ml) and cooled to 0° C. Sodium hydride (60% dispersion in oil, 200 mg, 3 mmol) was added, the mixture was stirred for 30 minutes, and then iodomethane (186 μl, 3 mmol) was added. The resulting suspension was stirred at RT for 18 hr. The reaction was quenched with water and the solvent was removed in vacuo. The residue was dissolved in ethyl acetate, washed with brine, then dried over Na₂SO₄. The solvent was removed in vacuo and the residue was purified by flash chromatography on silica gel (eluant 98% chloroform/2% methanol) to give a yellow gum identified as tert-butyl N-methyl-N-(2-picolyl)carbamate (100 mg, 19%).

### EXAMPLE 4

### tert-Butyl 4-aminomethyl-3-chlorobenzoate

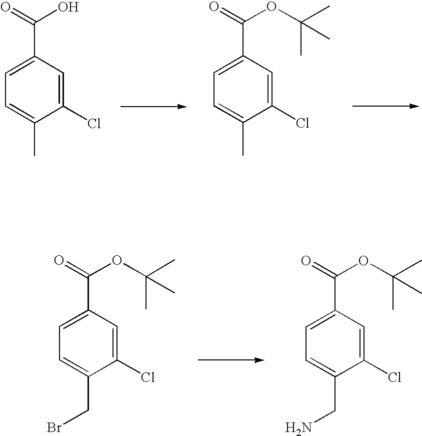

### 4A: tert-Butyl 3-chloro-4-methylbenzoate

Thionyl chloride (1 ml, 150 mmol) was added to a suspension of 3-chloro-4-methyl-benzoic acid (5.12 g, 30 mmol) in toluene (25 ml) and the mixture was heated at reflux for 2 hr. The solvent was removed in vacuo and the residue was azeotroped with toluene three times, then dissolved in anhydrous THF (40 ml) and cooled to 0° C. Lithium tert-butoxide (2.4 g, 30 mmol) was added and the mixture was stirred at RT for 3 days. Water (5 ml) was added and the solvent was removed in vacuo. The residue was dissolved in ethyl acetate. The solution was washed with 0.3M KHSO₄, sat. NaHCO₃ and brine, dried over Na₂SO₄ and concentrated in vacuo to give a pale yellow gum identified as tert-butyl 3-chloro-4-methylbenzoate (5.4 g, 79%).

### 4B: tert-Butyl 4-bromomethyl-3-chlorobenzoate

N-Bromosuccinimide (4.27 g, 24 mmol) and 2,2′-azo-bis(2-methylpropionitrile) (394 mg, 2.4 mmol) were added to a solution of tert-butyl 3-chloro-4-methylbenzoate (5.4 g, 23.8 mmol) in carbon tetrachloride (75 ml) and the mixture was heated at reflux for 18 hr. The solvent was removed in vacuo and the residue was purified by flash chromatography on silica gel (eluant 95% pet.ether/5% ethyl acetate) to give a white solid identified as tert-butyl 4-bromomethyl-3-chlorobenzoate (5.7 g, 78%).

### 4C: tert-Butyl 4-aminomethyl-3-chlorobenzoate

Ethanol (100 ml) was saturated with ammonia, then tert-butyl 4-bromomethyl-3-chloro-benzoate (5.7 g, 18.7 mmol) was added and the mixture was stirred at RT for 2 hr. The solvent was removed in vacuo and the residue was triturated with diethyl ether to give a white solid identified as tert-butyl 4-aminomethyl-3-chlorobenzoate (4.1 g, 91%).

### EXAMPLE 5

### 4-(tert-Butyloxycarbonylaminomethyl)-3-chlorobenzoic acid

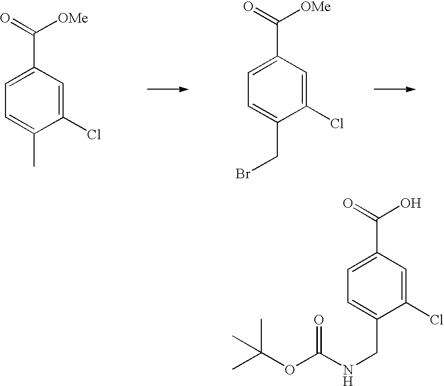

### 5A. Methyl 4-bromomethyl-3-chlorobenzoate

To a solution of methyl 3-chloro-4-methylbenzoate (5.0 g, 27.1 mmol) in carbon tetrachloride (50 ml) were added N-bromosuccinimide (5.8 g, 32.0 mmol) and 2,2′-azo-bis(2-methylpropionitrile) (0.442 g, 2.70 mmol). The mixture was heated at reflux for 18 hr, then allowed to cool to room temperature and concentrated in vacuo. The residue was purified by flash chromatography on silica (eluant pet. ether→5% ethyl acetate/95% pet. ether) to give an oil identified as methyl 4-bromomethyl-3-chlorobenzoate (5.96 g, 84%).

### 5B. 4-(tert-Butyloxycarbonylaminomethyl)-3-chlorobenzoic acid

To a saturated solution of ammonia in ethanol (170 ml) was added methyl 4-bromomethyl-3-chlorobenzoate from Example 5A (5.5 g, 20.9 mmol). The mixture was stirred at room temperature for 1 hr and then concentrated in vacuo. The residue was triturated with diethyl ether and the resultant white crystals were filtered off and washed with more diethyl ether. To a solution of this solid in water (100 ml) were added solutions of di-tert-butyl dicarbonate (5.0 g, 23.0 mmol) in dioxan (100 ml) and sodium hydroxide (1.86 g, 46.0 mmol) in water (100 ml). The mixture was stirred at room temperature for 18 hr and then concentrated in vacuo. The aqueous residue was acidified with citric acid and extracted with chloroform/2-propanol. The organic layer was washed with water, dried over MgSO₄, and concentrated in vacuo to give a white solid identified as 4-(tert-butyloxy-carbonylaminomethyl)-3-chlorobenzoic acid (2.8 g, 67%).

### EXAMPLE 6

### 4-(tert-Butyloxycarbonylaminomethyl)-3-nitrobenzoic acid

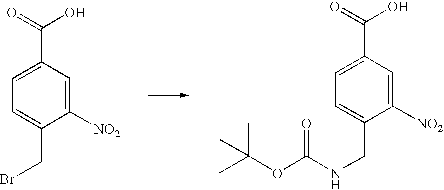

4-Bromomethyl-3-nitrobenzoic acid (4.75 g, 18.2 mmol) was reacted following the method of Example 5B to give a yellow solid identified as 4-(tert-butyloxycarbonylaminomethyl)-3-nitrobenzoic acid (2.6 g, 49%).

### EXAMPLE 7

### 4-Cyano-3-methylbenzoic acid

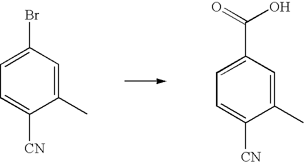

To a solution of 4-bromo-2-methylbenzonitrile (2.0 g, 10.2 mmol) in THF (100 ml) at −78° C. under a nitrogen atmosphere was added dropwise a 2.5M solution of n-butyl lithium (4.48 ml, 11.2 mmol). The mixture was stirred at −78° C. for 1 hr and then poured onto solid carbon dioxide (5 g) in THF (50 ml). The mixture was allowed to warm to room temperature. Water was added (200 ml) and the mixture was extracted with diethyl ether (3 times). The aqueous layer was acidified by addition of concentrated HCl and extracted with chloroform (3 times). The combined chloroform extracts were washed with water, dried over MgSO₄, and concentrated in vacuo to give a white solid identified as 4-cyano-3-methylbenzoic acid (1.2 g, 73%).

### EXAMPLE 8

### 4-Cyano-2-methylbenzoic acid

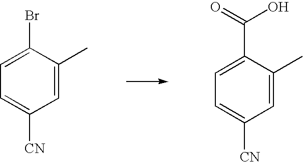

4-Bromo-3-methylbenzonitrile (2.0 g, 10.2 mmol) was reacted following the method of Example 7. The product was triturated with hexane to give a yellow solid identified as 4-cyano-2-methylbenzoic acid (0.96 g, 59%).

### EXAMPLE 9

### 4-(tert-Butyloxycarbonylaminomethyl)-2-fluorobenzoic acid

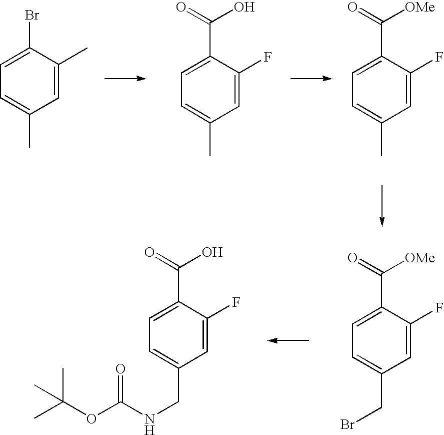

### 9A. 2-Fluoro-4-methylbenzoic acid

4-Bromo-3-fluorotoluene (8.33 g, 44.07 mmol) was reacted following the method of Example 7 to give a white solid identified as 2-fluoro-4-methylbenzoic acid (4.89 g, 72%).

### 9B. Methyl 2-fluoro-4-methylbenzoate

To a solution of 2-fluoro-4-methylbenzoic acid (6.04 g, 39.18 mmol) in toluene (80 ml) was added thionyl chloride (65 ml, 89.11 mmol). The mixture was heated at reflux for 2.5 hr, cooled and concentrated in vacuo. The residue was dissolved in dichloromethane (50 ml) and methanol (50 ml) was added. The mixture was stirred at room temperature for 2.5 hr and then concentrated in vacuo. The residue was dissolved in dichloromethane (100 ml), washed with saturated sodium bicarbonate solution and brine, dried over MgSO₄, and concentrated in vacuo to give a tan solid identified as methyl 2-fluoro-4-methylbenzoate (5.07 g, 77%).

### 9C. Methyl 4-bromomethyl-2-fluorobenzoate

Methyl 2-fluoro-4-methylbenzoate (5.07 g, 30.16 mmol) was reacted following the method of Example of 5A. The product was purified by flash chromatography on silica (eluant 20% ethyl acetate/80% pet. ether) to give an oil identified as methyl 4-bromomethyl-2-fluorobenzoate (5.9 g, 80%).

### 9D. 4-(tert-Butyloxycarbonylaminomethyl)-2-fluorobenzoic acid

Methyl 4-bromomethyl-2-fluorobenzoate (5.9 g, 24.13 mmol) was reacted following the method of Example 5B. The product was recrystallised from dioxan/pet. ether to give white crystals identified as 4-(tert-butyloxycarbonylaminomethyl)-2-fluorobenzoic acid (2.46 g, 38%).

### EXAMPLE 10

### 4-Cyano-3,5-dimethylbenzoic acid

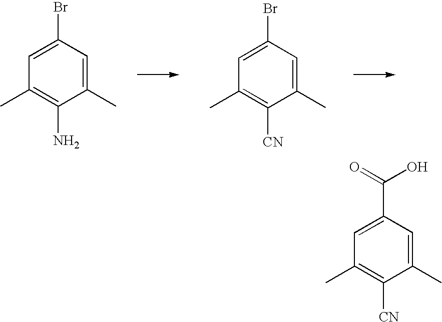

### 10A. 4-Bromo-2,6-dimethylbenzonitrile

4-Bromo-2,6-dimethylaniline (4.49 g, 22.4 mmol) was taken up in water (25 ml) and concentrated hydrochloric acid (8.0 ml) was added. The mixture was sonicated to form a fine suspension and then cooled to 0° C. A solution of sodium nitrite (1.67 g, 24.2 mmol) in water (5 ml) was then added dropwise so as to maintain the temperature of the reaction between 0-5° C. The mixture was stirred at 0-5° C. for 30 minutes and then neutralised by addition of solid sodium bicarbonate. The resulting solution was then added portionwise to a solution of copper cyanide (2.42 g, 27.0 mmol) and potassium cyanide (3.65 g, 56.1 mmol) in water (25 ml) at 70° C. The mixture was stirred at 70° C. for 30 minutes, allowed to cool and then extracted with toluene (2 times). The combined extracts were washed with water and brine, dried over MgSO₄, and concentrated in vacuo. The residue was purified by flash chromatography on silica (eluant 5% ethyl acetate/95% pet. ether) to give an orange solid identified as 4-bromo-2,6-dimethylbenzonitrile (3.2 g, 68%).

### 10B. 4-Cyano-3,5-dimethylbenzoic acid

4-Bromo-2,6-dimethylbenzonitrile (3.20 g, 15.2 mmol) was reacted following the method of Example 7 to give a tan solid identified as 4-cyano-3,5-dimethylbenzoic acid (1.5 g, 56%).

### EXAMPLE 11

### 4-Methyl-1-(N-(2-methyl-4-(2,3,4,5-tetrahydro-1,5-benzodiazepin-4-on-1-ylcarbonyl)benzylcarbamoyl)-L-thioprolyl)perhydro-1,4-diazepine

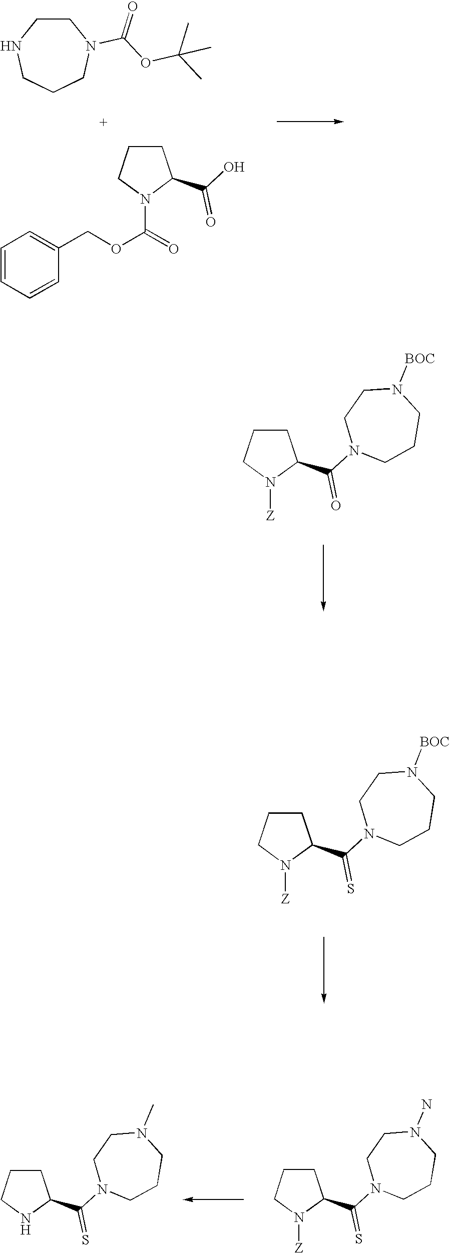

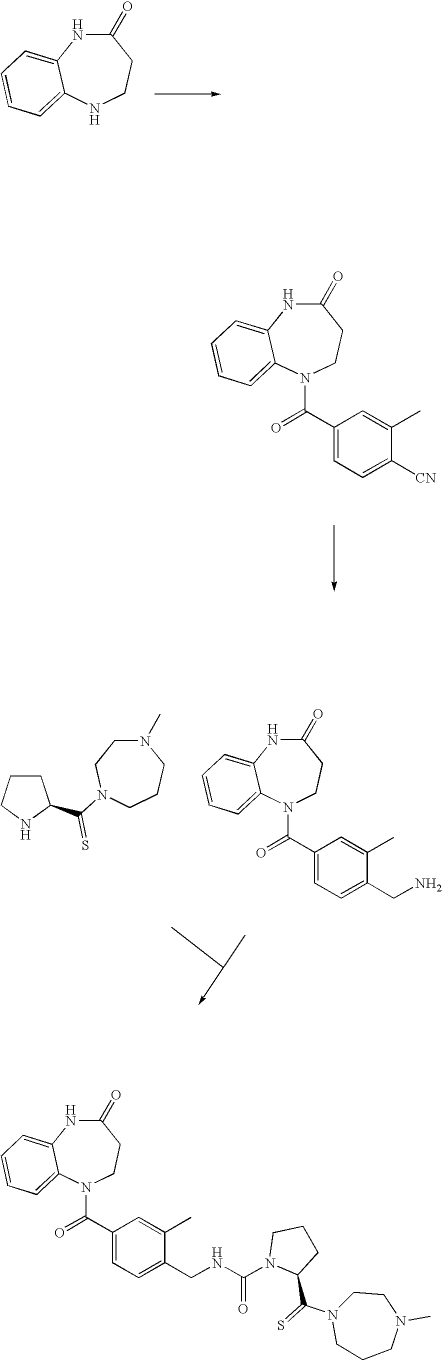

### 11A: 1-(N-Benzyloxycarbonyl-L-prolyl)-4-(tert-butyloxycarbonyl)perhydro-1,4-diazepine

HOBt (3.04 g, 20 mmol), WSCD (5.3 g, 26 mmol) and N-benzyloxycarbonyl-L-proline (4.7 g, 18.8 mmol) were added to a solution of tert-butyl homopiperazine-1-carboxylate (3.8 g, 18.8 mmol) and triethylamine (5.4 ml, 37.6 mmol) in dichloromethane (100 ml) at 0° C. The resulting solution was stirred at RT for 18 hr. The solvent was removed in vacuo and the residue was dissolved in ethyl acetate. The solution was washed with sat. NaHCO₃ and brine, dried over sodium sulphate and concentrated in vacuo to give a pale yellow gum identified as 1-(N-benzyloxycarbonyl-L-prolyl)-4-(tert-butyloxycarbonyl)perhydro-1,4-diazepine (8.1 g, 100%).

### 11B: 1-(N-Benzyloxycarbonyl-L-thioprolyl)-4-(tert-butyloxycarbonyl)perhydro-1,4-diazepine

Lawesson's reagent (6.79 g, 16.8 mmol) was added to a solution of 1-(N-benzyl-oxycarbonyl-L-prolyl)-4-(tert-butyloxycarbonyl)perhydro-1,4-diazepine (12 g, 28 mmol) in toluene (200 ml) and stirred at 90° C. for 1 hr then 85° C. for 18 hr. The solvent was removed in vacuo and the residue was purified by flash chromatography on silica gel (eluant 98% chloroform/2% methanol) to give 1-(N-benzyloxycarbonyl-L-thioprolyl)-4-(tert-butyloxycarbonyl)perhydro-1,4-diazepine (8.4 g, 67%).

MS [M+H]⁺447.3

### 11C: 1-(N-Benzyloxycarbonyl-L-thioprolyl)-4-methylperhydro-1,4-diazepine

A solution of 1-(N-benzyloxycarbonyl-L-thioprolyl)4-(tert-butyloxycarbonyl)perhydro-1,4-diazepine (8.4 g, 18.1 mmol) in 4N HCl/dioxan (20 ml) was stirred at RT for 35 min. The solvent was removed in vacuo and the residue was azeotroped with toluene. The resulting gum was dissolved in acetic acid/methanol (1:9, 100 ml) and stirred at 0° C. Formaldehyde (20% solution, 8.4 ml, 56 mmol) was added, the solution was stirred for 20 min, then sodium cyanoborohydride (2.35 g, 37 mmol) was added and the solution was stirred at RT for 18 hr. The solvent was removed in vacuo and the residue was purified by flash chromatography on silica gel (eluant 96% chloroform/3% methanol/1% triethylamine) to give a colourless gum identified as (6.56 g, 97%).

MS [M+H]⁺362.2

### 11D: 4-Methyl-1-L-thioprolylperhydro-1,4-diazepine

1-(N-Benzyloxycarbonyl-L-thioprolyl)-4-methylperhydro-1,4-diazepine (3 g, 8.3 mmol) was dissolved in ice-cold 30% HBr/acetic acid (20 ml). The resulting brown solution was stirred at RT for 3 hr, the solvent was removed in vacuo and the residue was azeotroped with toluene. Sat. NaHCO₃ (100 ml) was added until the solution showed a pH>8. The solution was washed with diethyl ether (2×40 ml) then evaporated in vacuo and azeotroped with toluene. The resulting solid was extracted with hot chloroform (3×100 ml) and the solvent was removed in vacuo to yield a brown gum identified as 4-methyl-1-L-thioprolylperhydro-1,4-diazepine (1.72 g, 91%).

MS [M+H]⁺228.1

### 11E: 1-(4-Cyano-3-methylbenzoyl)-2,3,4,5-tetrahydro-1,5-benzodiazepin-4-one

Thionyl chloride (0.4 ml, 6.0 mmol) was added to a suspension of 4-cyano-3-methyl-benzoic acid (322 mg, 2.0 mmol) in toluene (10 ml) and the mixture was heated at reflux for 90 min, then cooled to RT and concentrated in vacuo. The residue was azeotroped with toluene then dissolved in dichloromethane (5 ml) and added to a suspension of 2,3,4,5-tetrahydro-1,5-benzodiazepin-2-one (324 mg, 2.0 mmol) and triethylamine (280 μl, 2.0 mmol) in dichloromethane (3 ml). The mixture was stirred overnight at RT then concentrated in vacuo. The residue was partitioned between dichloromethane and 0.3M KHSO₄. The organic phase was washed with sat. NaHCO₃ and brine, dried over Na₂SO₄ and concentrated in vacuo to give a red solid identified as 1-(4-cyano-3-methylbenzoyl)-2,3,4,5-tetrahydro-1,5-benzodiazepin-4-one (600 mg, 98%).

### 11F: 1-(4-Aminomethyl-3-methylbenzoyl)-2,3,4,5-tetrahydro-1,5-benzodiazepin-4-one

Sodium borohydride (734 mg, 19.3 mmol) was added portionwise to an ice-cold suspension of 1-(4-cyano-3-methylbenzoyl)-2,3,4,5-tetrahydro-1,5-benzodiazepin-4-one (590 mg, 1.93 mmol) and CoCl₂.6H₂O (920 mg, 3.86 mmol) in methanol (10 ml). The mixture was stirred at RT for 1 hr, then quenched with 1M HCl and concentrated in vacuo. The aqueous residue was diluted with 1M HCl (50 ml) and filtered through Celite® filter agent. The filtrate was washed with diethyl ether (2×50 ml) then basified with 4M NaOH and extracted with chloroform (3×50 ml). The combined organic phases were dried over Na₂SO₄ and concentrated in vacuo to give a pale yellow solid identified as 1-(4-aminomethyl-3-methylbenzoyl)-2,3,4,5-tetrahydro-1,5-benzodiazepin-4-one (300 mg, 50%).

### 11G: 4-Methyl-1-(N-(2-methyl-4-(2,3,4,5-tetrahydro-1,5-benzodiazepin-4-on-1-ylcarbonyl)benzylcarbamoyl)-L-thioprolyl)perhydro-1,4-diazepine

1,1′-Carbonyldiimidazole (18 mg, 0.11 mmol) was added to a solution of 1-(4-aminomethyl-3-methylbenzoyl)-2,3,4,5-tetrahydro-1,5-benzodiazepin-4-one (31 mg, 0.1 mmol) and DIEA (54 μl, 0.3 mmol) in DMF (2 ml) under nitrogen gas and the solution was stirred at RT for 1 hr. A solution of 4-methyl-1-L-thioprolylperhydro-1,4-diazepine (22.7 mg, 0.1 mmol) and DIEA (54 μl, 0.3 mmol) in DMF (2 ml) was added and the solution was stirred at RT for 18 hr under nitrogen gas. The solvent was removed in vacuo and the residue was purified by flash chromatography on silica gel (eluant 97% chloroform/2% methanol/1% triethylamine) to give a white solid identified as 4-methyl-1-(N-(2-methyl-4-(2,3,4,5-tetrahydro-1,5-benzodiazepin-4-on-1-ylcarbonyl)benzylcarbamoyl)-L-thioprolyl)perhydro-1,4-diazepine (17 mg, 30%).

MS [M+H]⁺563.2

### EXAMPLE 12

### 4-Methyl-1-(N-(2-methyl-4-(1-methyl-4,10-dihydropyrazolo[5,4-b][1,5]benzodiazepin-5-ylcarbonyl)benzylcarbamoyl)-L-thioprolyl)perhydro-1,4-diazepine

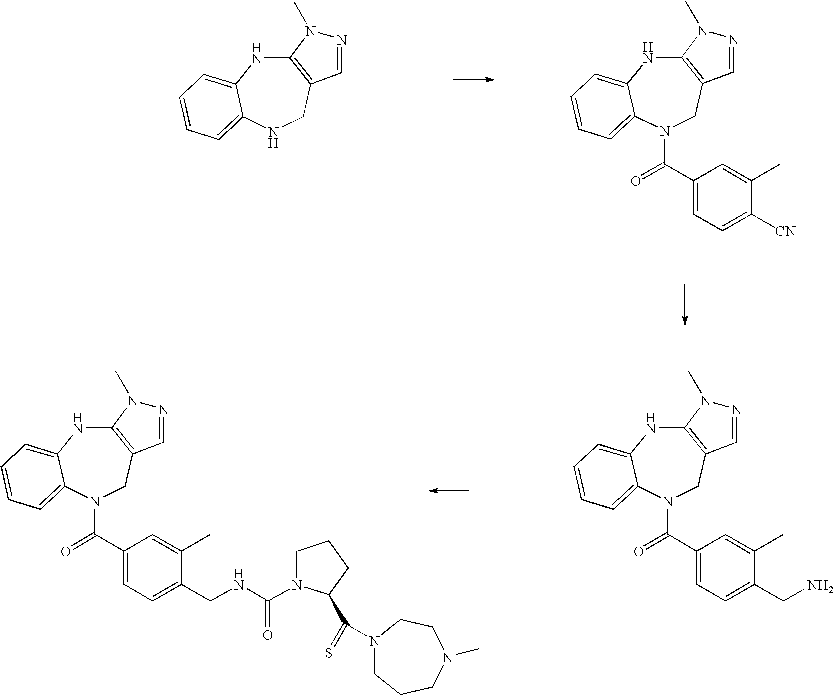

### 12A: 5-(4-Cyano-3-methylbenzoyl)-1-methyl-4,10-dihydropyrazolo[5,4-b][1,5]-benzodiazepine

Thionyl chloride (1.8 ml, 27 mmol) was added to a suspension of 4-cyano-3-methylbenzoic acid (1.29 g 8.0 mmol) in toluene (25 ml) and the mixture was heated at reflux for 2 hr, then cooled to RT and concentrated in vacuo. The residue was azeotroped with toluene then dissolved in dichloromethane (10 ml) and added to a suspension of 1-methyl-4,10-dihydropyrazolo[5,4-b][1,5]benzodiazepine (1.6 g, 8 mmol) and triethylamine (1.4 ml, 10 mmol) in dichloromethane (15 ml). The mixture was stirred overnight at RT then concentrated in vacuo. The residue was partitioned between chloroform and 0.3M KHSO₄. The aqueous phase was extracted with chloroform/2-propanol (80:20). The combined organic phases were washed with sat. NaHCO₃ and brine, dried over Na₂SO₄ and concentrated in vacuo. The residue was purified by flash chromatography on silica gel (eluant 95% chloroform/5% methanol) to give a pale yellow solid identified as 5-(4-cyano-3-methylbenzoyl)-1-methyl-4,10-dihydropyrazolo[5,4-b][1,5]benzodiazepine (2.4 g, 87%).

### 12B: 5-(4-Aminomethyl-3-methylbenzoyl)-1-methyl-4,10-dihydropyrazolo[5,4-b][1,5]-benzodiazepine

Sodium borohydride (1.27 g, 33.5 mmol) was added portionwise to an ice-cold suspension of 5-(4-cyano-3-methylbenzoyl)-1-methyl-4,10-dihydropyrazolo[5,4-b][1,5]benzodiazepine (1.15 g, 3.35 mmol) and CoCl₂.6H₂O (1.59 g, 6.7 mmol) in methanol (35 ml). The mixture was stirred at RT for 1 hr, then quenched with 1M KHSO₄ and concentrated in vacuo. The aqueous residue was diluted with 1M KHSO₄ (40 ml) and filtered through Celite® filter agent. The filtrate was washed with diethyl ether (2×50 ml) then basified with 2M NaOH and extracted with chloroform. The organic phase was dried over Na₂SO₄ and concentrated in vacuo to give a pale brown solid identified as 5-(4-aminomethyl-3-methylbenzoyl)-1-methyl-4,10-dihydropyrazolo[5,4-b][1,5]benzodiazepine (745 mg, 64%).

### 12C: 4-Methyl-1-(N-(2-methyl-4-(I -methyl-4,10-dihydropyrazolo[5,4-b][1,5]-benzodiazepin-5-ylcarbonyl)benzylcarbamoyl)-L-thioprolyl)perhydro-1,4-diazepine

1,1′-Carbonyldiimidazole (267 mg, 1.65 mmol) was added to a solution of 5-(4-aminomethyl-3-methylbenzoyl)-1-methyl-4,10-dihydropyrazolo[5,4-b][1,5]benzodiazepine (520 mg, 1.5 mmol) and DIEA (276 μl, 1.5 mmol) in DMF (10 ml) under nitrogen gas and the solution was stirred at RT for 1 hr. A solution of 4-methyl-1-L-thioprolylperhydro-1,4-diazepine (374 mg, 1.65 mmol) and DIEA (276 μl, 1.5 mmol) in DMF (10 ml) was added and the resulting solution was stirred at RT for 18 hr under nitrogen gas. The solvent was removed in vacuo and the residue was purified by flash chromatography on silica gel (eluant 96% chloroform/3% methanol/1% triethylamine then 92% chloroform/6% methanol/2% triethylamine) to yield a cream-coloured solid identified as 4-methyl-1-(N-(2-methyl-4-(1-methyl-4,10-dihydropyrazolo[5,4-b][1,5]benzodiazepin-5-ylcarbonyl)benzylcarbamoyl)-L-thioprolyl)perhydro-1,4-diazepine (568 mg, 63%).

NMR (270 MHz, CDCl₃) δ 1.91-2.77 (18H,m), 3.33-3.43 (2H,m), 3.62-3.64 (1H,m), 3.73 (3H,s), 3.75-3.95 (3H,m), 4.11-4.29 (2H,m), 4.55-4.57 (1H,m), 5.05-5.06 (1H,m), 5.85-5.91 (1H,m), 6.65-6.77 (3H,m), 6.94-6.98 (4H,m), 7.21-7.24 (1H,m) ppm. MS [M+H]⁺600.7

### EXAMPLE 13

### 4,4-Dimethyl-1-(N-(2-methyl-4-(1-methyl-4,10-dihydropyrazolo[5,4-b][1,5]-benzodiazepin-5-ylcarbonyl)benzylcarbamoyl)-L-thioprolyl)perhydro-1,4-diazepinium iodide

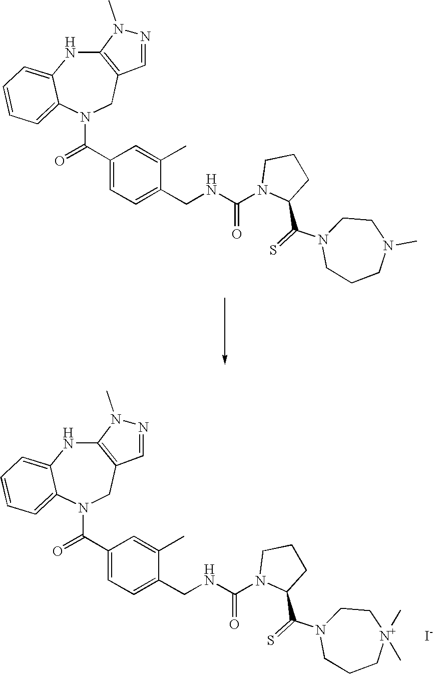

Iodomethane (13.9 mg, 6.1 μl, 0.098 mmol) was added to a solution of 4-methyl-1-(N-(2-methyl-4-(1-methyl-4,10-dihydropyrazolo[5,4-b][1,5]benzodiazepin-5-ylcarbonyl)benzylcarbamoyl)-L-thioprolyl)perhydro-1,4-diazepine (30 mg, 0.049 mmol) in THF (10 ml) and the mixture was stirred at RT for 4 hr. The resulting solid was collected and dissolved in water (5 ml). The solution was washed with ethyl acetate (2×10 ml) and lyophilized to give a white solid identified as 4,4-dimethyl-1-(N-(2-methyl-4-(1-methyl-4,10-dihydropyrazolo[5,4-b][1,5]benzodiazepin-5-ylcarbonyl)benzylcarbamoyl)-L-thioprolyl)perhydro-1,4-diazepinium iodide (29 mg, 79.6%). MS [M]⁺615.3

### EXAMPLE 14

### 4-Methyl-1-(N-(2-methyl-4-(5,6,7,8-tetrahydrothieno[3,2-b]azepin-4-ylcarbonyl)-benzylcarbamoyl)-L-thioprolyl)perhydro-1,4-diazepine

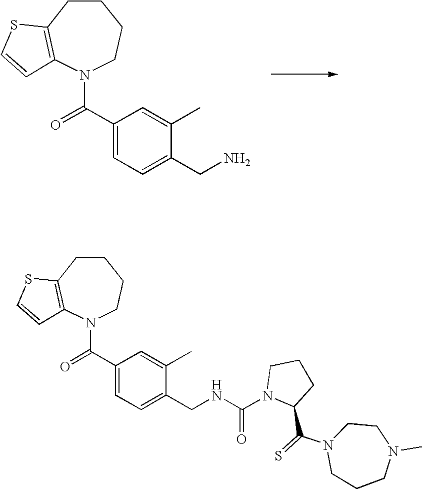

1,1′-Carbonyldiimidazole (20 mg, 0.12 mmol) was added to a solution of 4-(4-aminomethyl-3-methylbenzoyl)-5,6,7,8-tetrahydrothieno[3,2-b]azepine (36 mg, 0.1 mmol) and DIEA (50 μl, 0.3 mmol) in DMF (10 ml) under nitrogen gas and the solution was stirred at RT for 1 hr. A solution of 4-methyl-1-L-thioprolylperhydro-1,4-diazepine (34 mg, 0.15 mmol) and DIEA (50 μl, 0.3 mmol) in DMF (10 ml) was added and the resulting solution was stirred at RT for 18 hr under nitrogen gas. The solvent was removed in vacuo and the residue was purified by flash chromatography on silica gel (eluant 96% chloroform/3% methanol/1% triethylamine) and then by semi-preparative HPLC (Vydac C₁₈ column; 30%→80% 0.1%TFA/acetonitrile in 0.1%TFA/water over 40 min at 6 ml/min). Product-containing fractions were pooled and lyophilized to give a white solid identified as 4-methyl-1-(N-(2-methyl-4-(5,6,7,8-tetrahydrothieno[3,2-b]azepin-4-ylcarbonyl)benzylcarbamoyl)-L-thioprolyl)perhydro-1,4-diazepine trifluoroacetate (17 mg, 25%).

NMR (270 MHz, CD₃OD) δ 1.79-2.32 (10H,m), 2.71-2.99 (5H,m), 3.40-3.91 (10H,m), 4.26-4.53 (3H,m), 4.86-5.10 (2H,m), 6.27-6.28 (1H,m), 6.79-6.81 (1H,m), 7.13-7.41 (3H,m) ppm. MS [M+H]⁺574.2

### EXAMPLE 15

### 4-Methyl-1-(N-(2-methyl-4-(5,6,7,8-tetrahydrothieno[3,2-b]azepin-4-ylcarbonyl)-benzyloxycarbonyl)-L-prolyl)perhydro-1,4-diazepine

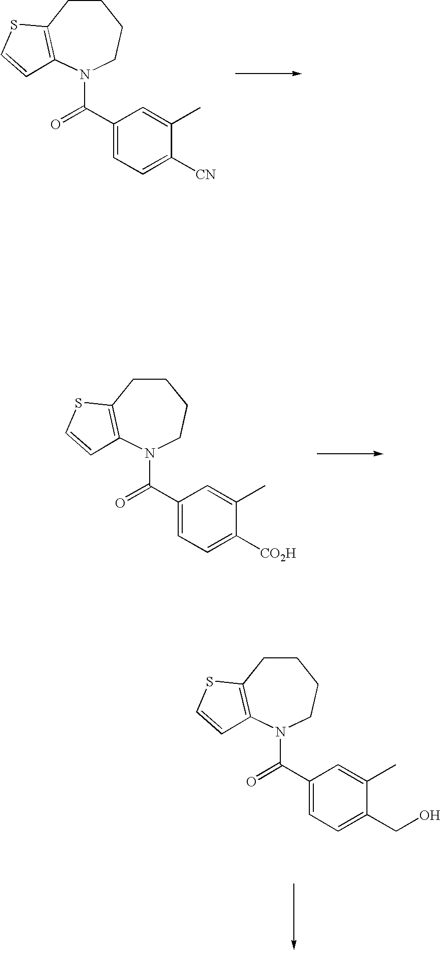

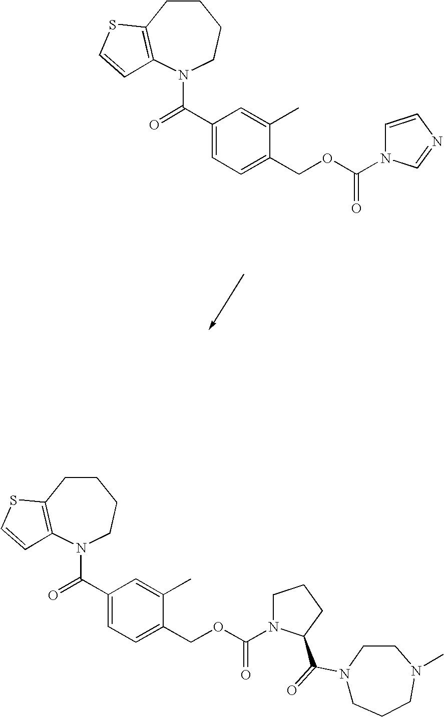

### 15A: 4-(4-Carboxy-3-methylbenzoyl)-5,6,7,8-tetrahydrothieno[3,2-b]azepine

A suspension of 4-(4-cyano-3-methylbenzoyl)-5,6,7,8-tetrahydrothieno[3,2-b]azepine (1 g, 3.3 mmol) in conc. sulphuric acid/water (1:1, 30 ml) was heated at reflux for 5 hr. The resulting solution was cooled to RT, diluted with water (20 ml) and extracted with chloroform (3×20 ml). The combined organic phases were extracted with sat. NaHCO₃ (2×20 ml). The combined aqueous extracts were acidified with 1M KHSO₄ and extracted with chloroform (3×20 ml). These chloroform extracts were combined, washed with brine, dried over Na₂SO₄ and concentrated in vacuo to give a pale brown solid identified as 4-(4-carboxy-3-methylbenzoyl)-5,6,7,8-tetrahydrothieno[3,2-b]azepine. (225 mg, 23%).

### 15B: 4-(4-Hydroxymethyl-3-methylbenzoyl)-5,6,7,8-tetrahydrothieno[3,2-b]azepine

Isobutyl chloroformate (250 μl, 2 mmol) was added to a solution of 4-(4-carboxy-3-methylbenzoyl)-5,6,7,8-tetrahydrothieno[3,2-b]azepine (470 mg, 1.48 mmol) and N-methylmorpholine (230 μl, 2.1 mmol) in THF (15 ml) at 0° C. and the mixture was stirred for 1 hr. The resultant suspension was filtered and the filtrate was added to a solution of sodium borohydride (131 mg, 3.45 mmol) in water (15 ml) at 0° C. The solution was stirred at RT for 2 hr, then sat. NH₄Cl (5 ml) was added and the THF was removed in vacuo. The remaining solution was diluted with water and extracted with chloroform (3×20 ml). The combined organic phases were washed with brine, dried over Na₂SO₄ and concentrated in vacuo to give a pale brown solid identified as 4-(4-hydroxymethyl-3-methylbenzoyl)-5,6,7,8-tetrahydrothieno[3,2-b]azepine (330 mg, 74%).

### 15C: 4-(4-(1-Imidazolecarbonyloxymethyl)-3-methylbenzoyl)-5,6,7,8-tetrahydrothieno[3,2-b]azepine

1,1′-Carbonyldiimidazole (36 mg, 0.22 mmol) was added to a solution of 4-(4-hydroxymethyl-3-methylbenzoyl)-5,6,7,8-tetrahydrothieno[3,2-b]azepine (60 mg, 0.17 mmol) in DMF (2 ml) under nitrogen gas and the solution was stirred at RT for 18 hr. The solvent was removed in vacuo and the residue was purified by flash chromatography on silica gel (eluant 97% chloroform/3% methanol) to give a colourless gum identified as 4-(4-(1-imidazolecarbonyloxymethyl)-3-methylbenzoyl)-5,6,7,8-tetrahydrothieno[3,2-b]azepine (60 mg, 45%).

### 15D: 4-Methyl-1-(N-(2-methyl-4-(5,6,7,8-tetrahydrothieno[3,2-b]azepin-4-ylcarbonyl)-benzyloxycarbonyl)-L-prolyl)perhydro-1,4-diazepine

A solution of 4-(4-(1-imidazolecarbonyloxymethyl)-3-methylbenzoyl)-5,6,7,8-tetrahydrothieno[3,2-b]azepine (27.24 mg, 0.12 mmol), 4-methyl-1-L-prolylperhydro-1,4-diazepine (47 mg, 0.12 mmol) and DIEA (45 μl, 0.125 mmol) in ethyl acetate (20 ml) was heated at reflux for 48 Hrs. The solvent was removed in vacuo and the residue was purified by flash chromatography on silica gel (eluant 96% chloroform/3% methanol/1% triethylamine) then by semi-preparative HPLC (Vydac C₁₈ column; 25%→65% 0.1%TFA/acetonitrile in 0.1%TFA/water over 40 min at 6 ml/min). Product-containing fractions were pooled and lyophilized to give a white solid identified as 4-methyl-1-(N-(2-methyl-4-(5,6,7,8-tetrahydrothieno[3,2-b]azepin-4-ylcarbonyl)benzyloxycarbonyl)-L-prolyl)perhydro-1,4-diazepine trifluoroacetate (11 mg, 13%).

NMR (270 MHz, CD₃OD) δ 1.80-2.46 (15H,m), 2.92-2.98 (5H,m), 3.29-3.79 (11H,m), 4.07-4.15 (1H,m), 4.84-5.13 (3H,m), 6.21-6.25 (1H,m), 6.75-6.78 (1H,m), 7.02-7.21 (3H,m) ppm. MS [M+H]⁺585.3.

### EXAMPLE 16

### (4R)-N^{α}-(2-Chloro-4-(5,6,7,8-tetrahydrothieno[3,2-b]azepin-4-ylcarbonyl)-benzylcarbamoyl)-4methoxy-L-proline-N-methyl-N-(2-picolyl)amide

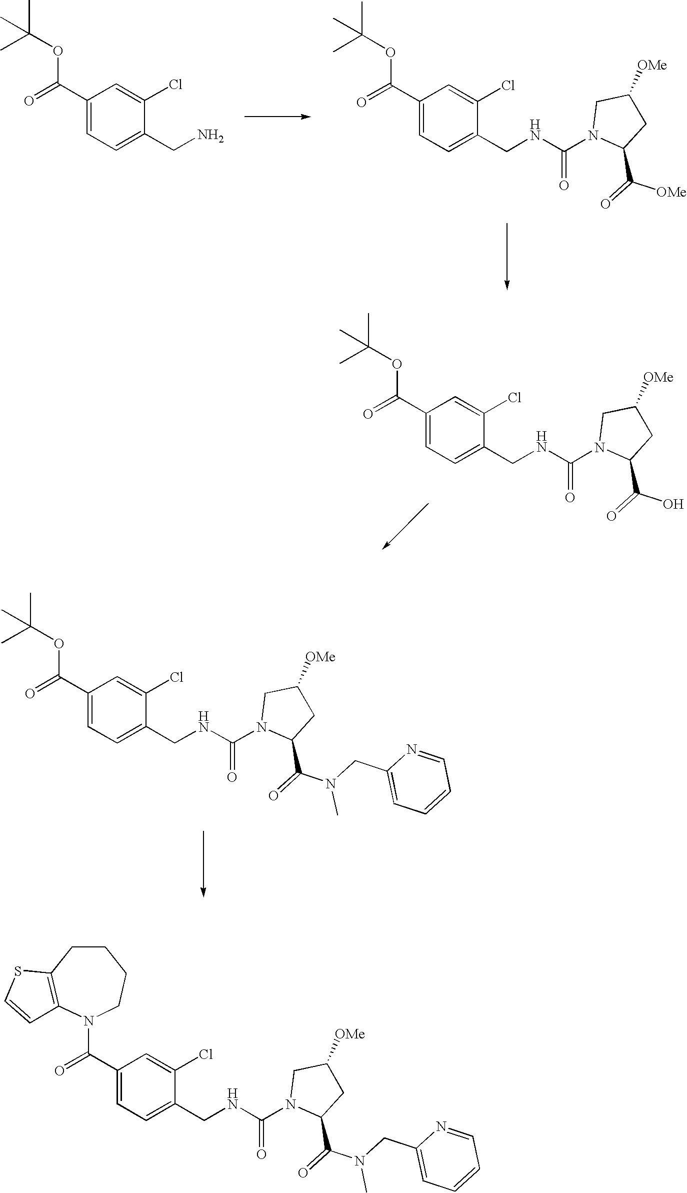

### 16A: (4R)-N^{α}-(4-(tert-Butyloxycarbonyl)-2-chlorobenzylcarbamoyl)4-methoxy-L-proline methyl ester

1,1′-Carbonyldiimidazole (800 mg, 4.8 mmol) was added to a solution of tert-butyl 4-(aminomethyl)-3-chlorobenzoate (967 mg, 4 mmol) and DIEA (1 ml, 6 mmol) in DMF (30 ml) under nitrogen gas and the solution was stirred at RT for 1 hr. A solution of (4R)4-methoxy-L-proline methyl ester (663 mg, 4.17 mmol) in DMF (10 ml) was added and the solution was stirred at RT for 18 hr. The solvent was removed in vacuo and the residue was dissolved in ethyl acetate. The solution was washed with 0.3M KHSO₄, sat. NaHCO₃ and brine, dried over Na₂SO₄, and concentrated in vacuo. The residue was purified by flash chromatography on silica gel (eluant 80% pet.ether/20% ethyl acetate) to give a white sold identified as (940 mg, 55%).

### 16B: (4R)-N^{α}-(4-(tert-Butyloxycarbonyl)-2-chlorobenzylcarbamoyl)-4-methoxy-L-proline

Lithium hydroxide (139 mg, 3.3 mmol) was added to a solution of (4R)-N^{α}-(4-(tert-butyloxycarbonyl)-2-chlorobenzylcarbamoyl)4-methoxy-L-proline methyl ester (930 mg, 2.18 mg) in dioxan/water (1:1, 30 ml) and the mixture was stirred at RT for 1 hr. The solvent was removed in vacuo and the residue was partitioned between ethyl acetate and 0.3M KHSO₄. The aqueous layer was extracted with ethyl acetate (2×30 ml). The organic phases were combined and washed with brine, dried over Na₂SO₄ and concentrated in vacuo to give a white solid identified as (4R)-N^{α}-(4-(tert-butyloxycarbonyl)-2-chlorobenzylcarbamoyl)-4-methoxy-L-proline (890 mg, 99%).

### 16C: (4R)-N^{α}-(4-(tert-Butyloxycarbonyl)-2-chlorobenzylcarbamoyl)-4-methoxy-L-proline-N-methyl-N-(2-picolyl)amide

tert-Butyl N-methyl-N-(2-picolyl)carbamate (100 mg, 0.45 mmol) was dissolved in 4N HCl/dioxan (10 ml) and the solution was stirred at RT for 1 hr. The solvent was removed in vacuo and the residue was azeotroped with toluene. The resulting gum was dissolved in dichloromethane (5 ml) and triethylamine (140 μl, 1 mmol) was added to give a solution of N-methyl-2-picolylamine.

In a second flask, HOBt (107 mg, 0.7 mmol) and WSCD (106 mg, 0.55 mmol) were added to a solution of (4R)-N^{α}-(4-(tert-butyloxycarbonyl)-2-chlorobenzylcarbamoyl)4-methoxy-L-proline (185 mg, 0.45 mmol) in dichloromethane (10 ml) at 0° C. The resulting solution was allowed to warm to RT and stirred for 30 min. The solution of N-methyl-2-picolylamine was added to this solution and the reaction mixture was stirred at RT for 18 hr. The solvent was removed in vacuo and the residue was dissolved in ethyl acetate. The solution was washed with 0.3M KHSO₄, sat. NaHCO₃ and brine, dried over Na₂SO₄ and concentrated in vacuo. The residue was purified by flash chromatography on silica gel (eluant 96% chloroform/4% methanol) to give a white gum identified as (4R)-N^{α}-(4-(tert-butyloxycarbonyl)-2-chlorobenzylcarbamoyl)-4-methoxy-L-proline-N-methyl-N-(2-picolyl)amide (170 mg, 73%).

### 16D: (4R)-N^{α}-(2-Chloro-4-(5,6,7,8-tetrahydrothieno[3,2-b]azepin-4-ylcarbonyl)-benzylcarbamoyl)-4-methoxy-L-proline-N-methyl-N-(2-picolyl)amide

(4R)-N^{α}-(4-(tert-Butyloxycarbonyl)-2-chlorobenzylcarbamoyl)-4-methoxy-L-proline-N-methyl-N-(2-picolyl)amide (60 mg, 0.12 mmol) was dissolved in 60% TFA/DCM (10 ml) and the solution was stirred at RT for 90 min. The solvent was removed in vacuo, and the residue was dissolved in dichloromethane (10 ml) then cooled to 0° C. 5,6,7,8-Tetrahydrothieno[3,2-b]azepine (20 mg, 0.12 mmol), WCSD (48 mg, 0.25 mmol), 4-dimethylaminopyridine (15 mg, 0.12 mmol) and triethlyamine (56 μl, 0.4 mmol) were added and the solution was heated at reflux for 1 hr. The solvent was removed in vacuo and the residue was dissolved in ethyl acetate. The solution was washed with 0.3M KHSO₄, sat. NaHCO₃ and brine, dried over Na₂SO₄ and concentrated in vacuo. The residue was purified by flash chromatography on silica gel (eluant 97% chloroform/3% methanol) to give a white solid identified as (4R)-N^{α}-(2-chloro-4-(5,6,7,8-tetrahydrothieno[3,2-b]azepin-4-ylcarbonyl)benzylcarbamoyl)-4-methoxy-L-proline-N-methyl-N-(2-picolyl)amide (32 mg, 45%)

NMR (270 MHz CDCl₃) δ 1.74-2.26 (8H,m), 2.86-2.89 (3H,m), 3.07-3.32 (4H,m), 3.44-4.97 (10H,m), 6.12-6.16 (1H,m), 6.58-6.65 (1H,m), 6.99-7.28 (4H,m), 7.53-7.70 (2H,m), 8.17-8.44 (1H,m) ppm. MS [M+H]⁺596.2

### EXAMPLE 17

### 1-((4R)-N^{α}-(2-Chloro-4-(5,6,7,8-tetrahydrothieno[3,2-b]azepin-4-ylcarbonyl)-benzylcarbamoyl)-4-methoxy-L-prolyl)-4-(1-pyrrolidinyl)piperidine

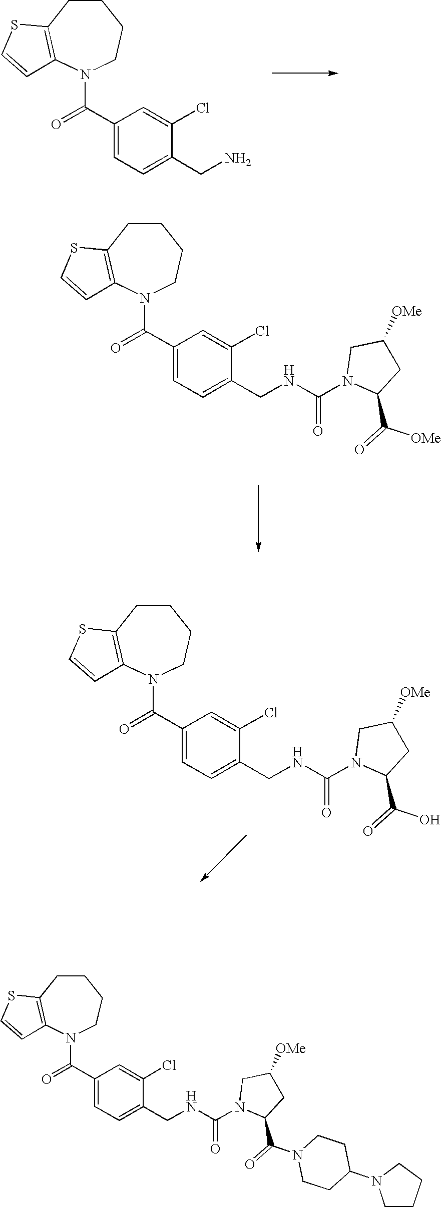

### 17A: (4R)-N^{α}-(2-Chloro-4-(5,6,7,8-tetrahydrothieno[3,2-b]azepin-4-ylcarbonyl)-benzylcarbamoyl)-4-methoxy-L-proline methyl ester

1,1′-Carbonyldiimidazole (197.8 mg,1.22 mmol) was added to a solution of 4-(4-aminomethyl-3-chlorobenzoyl)-5,6,7,8-tetrahydrothieno[3,2-b]azepine (400 mg, 1.11 mmol) and DIEA (306 l, 1.66 mmol) in DMF (10 ml) under nitrogen gas and the solution was stirred at RT for 1 hr. A solution of (4R)-4-methoxy-L-proline methyl ester (176 mg, 1.11 mmol) in DMF (10 ml) was added and the mixture was stirred at RT for 18 hr under nitrogen gas. The solvent was removed in vacuo and the residue was purified by flash chromatography on silica gel (eluant 98% chloroform/1% methanol/1% acetic acid) to give a white solid identified as (4R)-N^{α}-(2-chloro-4-(5,6,7,8-tetrahydrothieno[3,2-b]azepin-4-ylcarbonyl)benzylcarbamoyl)4-methoxy-L-proline methyl ester (550 mg, 98%).

### 17B: (4R)N^{α}-(2-Chloro-4-(5,6,7,8-tetrahydrothieno[3,2-b]azepin-4-ylcarbonyl)-benzylcarbamoyl)-4-methoxy-L-proline

A solution of lithium hydroxide (68.5 mg, 1.63 mmol) and (4R)-N^{α}-(2-chloro-4-(5,6,7,8-tetrahydrothieno[3,2-b]azepin-4-ylcarbonyl)benzylcarbamoyl)-4-methoxy-L-proline methyl ester (555 mg, 1.08 mmol) in THF/water (1:1, 40 ml) was stirred at RT for 1 hr. The dioxan was removed in vacuo and the aqueous residue was acidified with 1 N HCl and extracted with ethyl acetate (3×50 ml). The organic extracts were combined and washed with water, dried over Na₂SO₄ and concentrated in vacuo to give a white solid identified as (4R)-N^{α}-(2-chloro-4-(5,6,7,8-tetrahydrothieno[3,2-b]azepin-4-ylcarbonyl)-benzylcarbamoyl)-4-methoxy-L-proline (490 mg, 92%).

### 17C: 1-((4R)-N^{α}-(2-Chloro-4-(5,6,7,8-tetrahydrothieno[3,2-b]azepin-4-ylcarbonyl)-benzylcarbamoyl)-4-methoxy-L-prolyl)-4-(1-pyrrolidinyl)piperidine

HOBt (14.8 mg, 0.097 mmol), WSCD (22.68 mg, 0.11 mmol) and 4-(pyrrolidinyl)piperidine (13.7 mg, 0.089 mmol) were added to an ice-cold solution of (4R)-N^{α}-(2-chloro-4-(5,6,7,8-tetrahydrothieno[3,2-b]azepin-4-ylcarbonyl)benzylcarbamoyl)4-methoxy-L-proline (40 mg, 0.081 mmol) and triethylamine (18 μl, 0.12 mmol) in dichloromethane (10 ml). The resulting solution was stirred at RT for 18 hr. The solvent was removed in vacuo and the residue was dissolved in ethyl acetate. The solution was washed with sat. NaHCO₃ and brine, dried over Na₂SO₄ and concentrated in vacuo. The residue was purified by flash chromatography on silica gel (eluant 94% chloroform/4% methanol/2% triethylamine) to give a white solid identified as 1-((4R)-N^{α}-(2-chloro-4-(5,6,7,8-tetrahydrothieno[3,2-b]azepin-4-ylcarbonyl)benzylcarbamoyl)4-methoxy-L-prolyl)-4-(1-pyrrolidinyl)piperidine (40.9 mg, 80%).

NMR (270 MHz CDCl₃) δ 1.11-1.33(7H,m), 1.76-2.15 (12H,m), 2.60-2.73(3H,m), 2.88-2.92 (2H,m), 3.28 (3H,s), 3.29-3.32 (1H,m), 3.67-3.70 (1H,m), 3.95-4.23 (3H,m), 4.61-4.83 (2H,m), 4.76-4.78 (1H,m), 4.86-4.89 (1H,m), 6.16-6.17 (1H,m), 6.65-6.67 (1H,m), 7.02-7.05 (1H,m), 7.24-7.26 (3H,m) ppm. MS [M+H]⁺628.3

The following compounds are prepared by analogous methods.

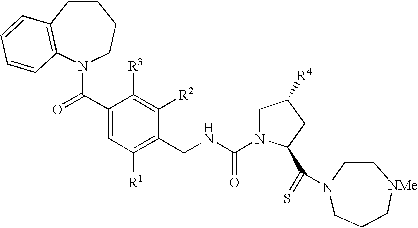

| Example | | | | | |
|---|---|---|---|---|---|
| No. | R¹ | R² | R³ | R⁴ | [M + H]⁺ |
| 18 | H | H | Me | H | 548.3 |
| 19 | H | H | Me | OMe | 578.4 |
| 20 | Me | Me | H | H | 562.4 |
| 21 | Me | Me | H | OMe | 592.4 |
| 22 | Et | H | H | H | 562.3 |
| 23 | Et | H | H | OMe | 592.3 |
| 24 | Cl | H | H | H | 568.3 |
| 25 | Cl | H | H | OMe | 598.3 |
| 26 | H | H | H | H | 534.4 |
| 27 | H | H | H | OMe | 564.4 |

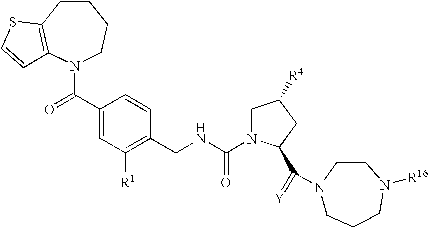

| Example | | | | | |
|---|---|---|---|---|---|
| No. | R¹ | R⁴ | R¹⁶ | Y | [M + H]⁺ |
| 28 | Cl | OMe | Me | S | 604.2 |
| 29 | F | H | Me | S | 558.2 |
| 30 | Me | H | Me | S | 554.2 |
| 31 | Me | H | Pr | S | 582.3 |
| 32 | Me | H | iBu | S | 596.3 |
| 33 | Me | H | Et | S | 568.3 |
| 34 | Cl | H | Me | O | 558.3 |
| 35 | Cl | OMe | Me | O | 588.4 |

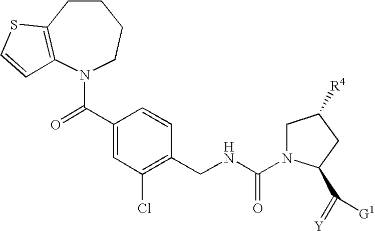

| Example | | | | |
|---|---|---|---|---|
| No | R⁴ | G¹ | Y | [M + H]⁺ |
| 36 37 | H OMe | 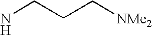 | O O | 546.3 576.2 |
| 38 39 | H OMe | 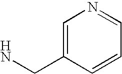 | O O | 552.2 582.2 |
| 40 41 42 | H OMe OMe | 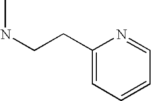 | O O S | 580.2 610.3 626.2 |
| 43 44 | OMe H | 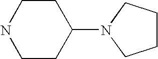 | S O | 644.2 598.2 |
| 45 56 | H OMe | 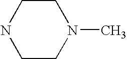 | O O | 544.2 574.2 |

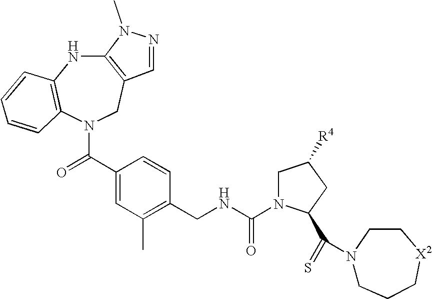

| Example | | | |
|---|---|---|---|
| No | R⁴ | X² | [M + H]⁺ |
| 47 | H | N—H | 587.3 |
| 48 | H | N-Pr | 629.2 |
| 49 | H | N-iBu | 643.3 |
| 37 | H | N-Et | 615.1 |
| 50 | OMe | N-Me | 631.3 |

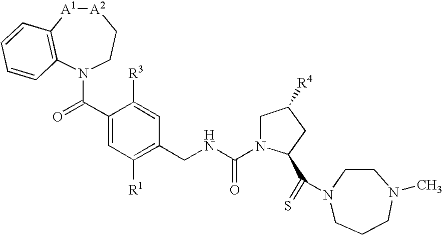

| Example | | | | | | |
|---|---|---|---|---|---|---|
| No | R¹ | R³ | R⁴ | A¹ | A² | [M + H]⁺ |
| 51 | H | Me | H | NH | CH₂ | 549.3 |
| 52 | H | Me | OMe | NH | CH₂ | 579.3 |
| 53 | Me | H | H | N-Et | CH₂ | 577.4 |
| 54 | Me | H | OMe | N-Et | CH₂ | 607.3 |
| 55 | H | Me | H | N-Me | CH₂ | 563.4 |
| 56 | H | Me | OMe | N-Me | CH₂ | 593.3 |
| 57 | Me | H | H | N-Me | CH₂ | 563.4 |
| 58 | Me | H | OMe | N-Me | CH₂ | 593.4 |
| 59 | Me | H | H | N—(CH₂)₂OH | CH₂ | 593.4 |
| 60 | Me | H | OMe | N—(CH₂)₂OH | CH₂ | 623.4 |
| 61 | Me | H | H | S | CH₂ | 566.3 |
| 62 | Me | H | OMe | S | CH₂ | 596.3 |
| 63 | H | H | H | O | CH₂ | 536.4 |
| 64 | H | H | OMe | O | CH₂ | 566.4 |
| 65 | Cl | H | H | O | CH₂ | 570.3 |
| 66 | Cl | H | OMe | O | CH₂ | 600.3 |
| 67 | Me | H | H | CH₂ | NH | 549.3 |

| | | | |
|---|---|---|---|
| Example | | | |
| No | R⁴ | G² | [M + H]⁺ |

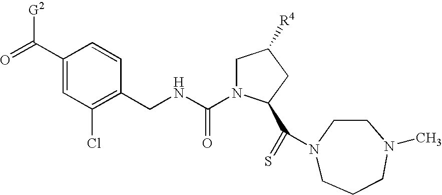

| | | | |
|---|---|---|---|
| 68 69 | H OMe | 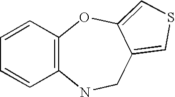 | 624.2 654.3 |
| 70 71 | H OMe | 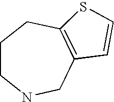 | 574.2 604.2 |

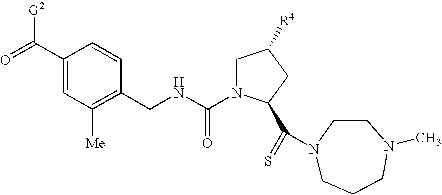

| | | | |
|---|---|---|---|
| 72 73 | H OMe | 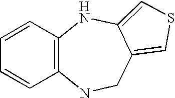 | 603.3 N/A |
| 74 75 | H OMe | 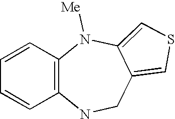 | 617.3 647.3 |
| 76 77 | H OMe | 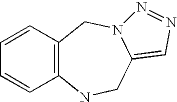 | 587.3 617.3 |
| 78 | H | 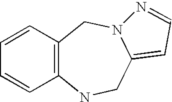 | 586.3 |
| 79 | H | 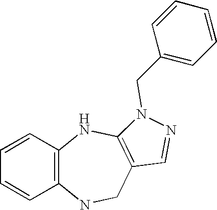 | 677.3 |
| 80 | H | 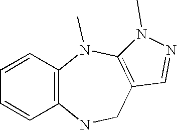 | 615.2 |
| 81 | H | 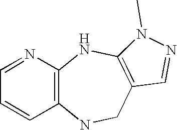 | 602.2 |
| 82 | H | | 598.4 |
| 83 | OMe | 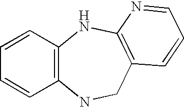 | 628.4 |
| 84 | H | 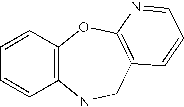 | 599.2 |
| 85 86 | H OMe | 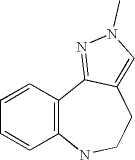 | 600.4 630.4 |

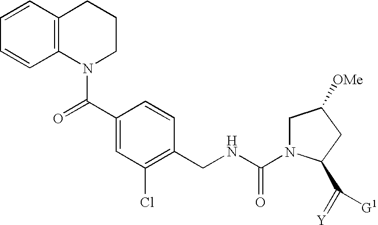

| Example | | | |
|---|---|---|---|
| No | Y | G¹ | [M + H]⁺ |
| 87 88 | S O | 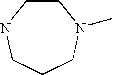 | 584.3 |
| 89 | S | 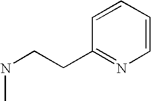 | 606.3 |

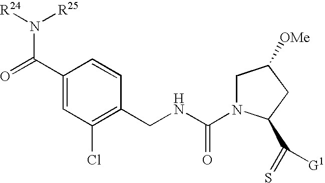

| Ex- | | | | | |
|---|---|---|---|---|---|
| am- | | | | | |
| ple | | | | | [M + |
| No | R²⁴ | R²⁵ | Y | G¹ | H]⁺ |
| 90 91 | i-Bu i-Pr | i-Bu 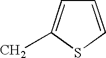 | S S | 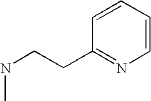 | 602.3 628.3 |
| 92 93 94 | Me Et Et | i-Pr i-Pr i-Pr | S O S | 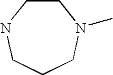 | 524.4 522.4 |
| 95 96 97 | i-Pr i-Pr i-Bu | i-Pr 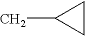 i-Bu | S S S | 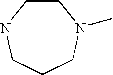 | 552.4 564.4 580.2 |
| 98 99 100 | n-Pn n-Pn i-Pr | n-Pn n-Pn Ph | O S O | 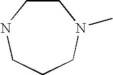 | 570.3 |
| 101 102 103 | n-Bu Et i-Pr | Ph 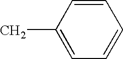 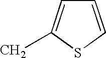 | O S O | 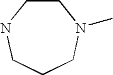 | 590.3 |
| 104 105 | i-Pr Ph | 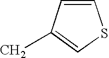 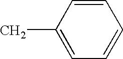 | S O | 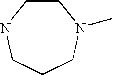 | |

### EXAMPLE 106

### In vitro Testing

Compounds were assayed to determine their ability to inhibit the binding of [¹²⁵I]OVA to a cell membrane preparation of OT receptors (binding assay) and to mimic the cellular consequences of OT stimulation on intact cells (functional assay). In the binding assay the compounds of the invention generally demonstrate significant inhibition of radioligand binding at concentrations of 50 μM or less. In the functional assay, the compounds of the invention cause significant cellular activation at concentrations of 30 μM or less. Preferred compounds cause significant activation at concentrations of 300 nM or less and can induce the same maximal effect as OT. The compounds are either significantly less active or completely devoid of activity in assays for vasopressin-like activity.

### EXAMPLE 107

### In vivo Testing

Representative compounds were tested for activity in the rat uterine contractility model, which is a recognised test for OT agonism. The compounds increased the strength and frequency of the uterine contractions at doses below 50 mg/kg. Selected compounds were then given either i.c.v. or i.v. to male rats and the erectile response was determined.

### EXAMPLE 108

### Tablet for Oral Administration

Tablets containing 100 mg of the compound of Example 11 as the active agent are prepared from the following:

| | |
|---|---|
| Compound of Example 11 | 200.0 g |
| Corn starch | 71.0 g |
| Hydroxypropylcellulose | 18.0 g |
| Carboxymethylcellulose calcium | 13.0 g |
| Magnesium stearate | 3.0 g |
| Lactose | 195.0 g |
| Total | 500.0 g |

The materials are blended and then pressed to give 2000 tablets of 250 mg, each containing 100 mg of the compound of Example 11.

The foregoing demonstrates that the compounds according to the present invention act as agonists at the oxytocin receptor and accordingly they may find utility as pharmaceutical agents for the treatment of conditions such as sexual disorders including male erectile dysfunction and ejaculatory disorders, female sexual dysfunction, cancer of the prostate, breast, ovary and bones, osteoporosis, benign prostatic hyperplasia, post-partum bleeding, and depression. The compounds may also be used to induce labour or delivery of the placenta, to decrease arterial blood pressure, to decrease exaggerated responses to stress and to increase the nociceptive threshold.

The scope of the present invention is further defined in the following claims.

1. A compound according to general formula 1, or a pharmaceutically acceptable salt thereof 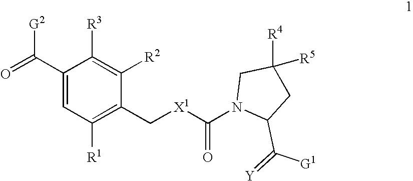
wherein:
G¹ is selected from a group according to general formula 2 and a group according to general formula 3; 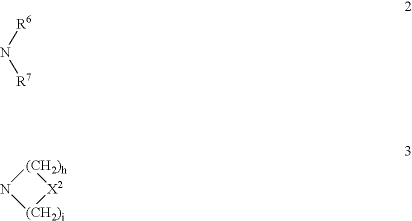
G² is selected from a group according to general formula 4, a group according to general formula 5, a group according to general formula 6, a group according to general formula 7, a group according to general formula 8 and a group according to general formula 9; 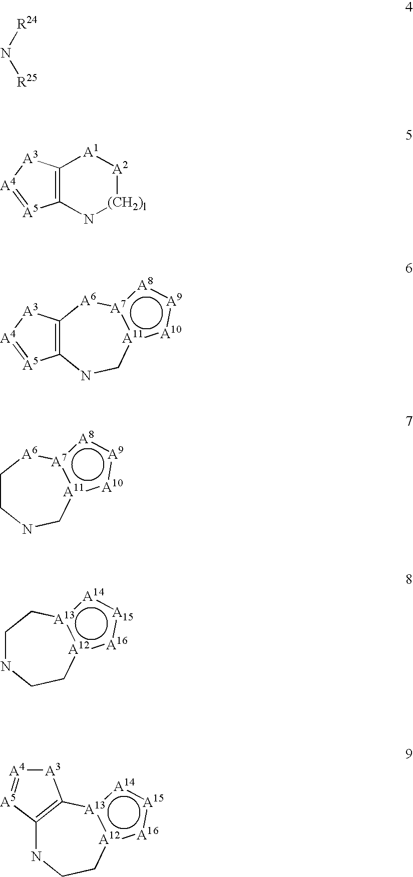
A¹ is selected from CH₂, CH(OH), NH, N-alkyl, N—(CH₂)ₙ-R²⁷, O and S;
A² is selected from CH₂, CH(OH), C(═O) and NH;
A³ is selected from S, NH, N-alkyl, —CH═CH— and —CH═N—;
A⁴ and A⁵ are each selected from CH and N;
A⁶ is selected from CH₂, NH, N-alkyl and O;
A⁷ and A¹¹ are selected from C and N;
A⁸ and A⁹ are selected from CH, N, NH, N(CH₂)ₘR²⁶ and S;
A¹⁰ is selected from —CH═CH—, CH, N, NH, N(CH₂)ₘR²⁶ and S;
A¹² and A¹³ are selected from N and C;
A¹⁴, A¹⁵ and A¹⁶ are selected from NH, N—CH₃, S, N and CH;
X¹ is selected from O and NH;
X² is selected from NR¹⁶, CH—NR¹⁷R¹⁸, CH—CH₂NR¹⁷R¹⁸, N⁺R¹⁹R²⁰, CH—N⁺R²¹R²²R²³ and CH—CH₂N^{+R}²¹R²²R²³;
Y is selected from O and S;
R¹, R² and R³ are each selected from H, alkyl, O-alkyl, F, Cl and Br;
R⁴ and R⁵ are each selected from H, O-alkyl, O-benzyl and F, or R⁴ and R⁵ together are ═O, —O(CH₂)ₐO— or —S(CH₂)ₐS—;
R⁶ is selected from a group according to general formula 10, a group according to general formula 11, a group according to general formula 12, a group according to general formula 13, a group according to general formula 14, a group according to general formula 15, a group according to general formula 16, a group according to general formula 17, a group according to general formula 18, a group according to general formula 19, a group according to general formula 20, a group according to general formula 21, a group according to general formula 22, a group according to general formula 23, a group according to general formula 24 and a group according to general formula 25; 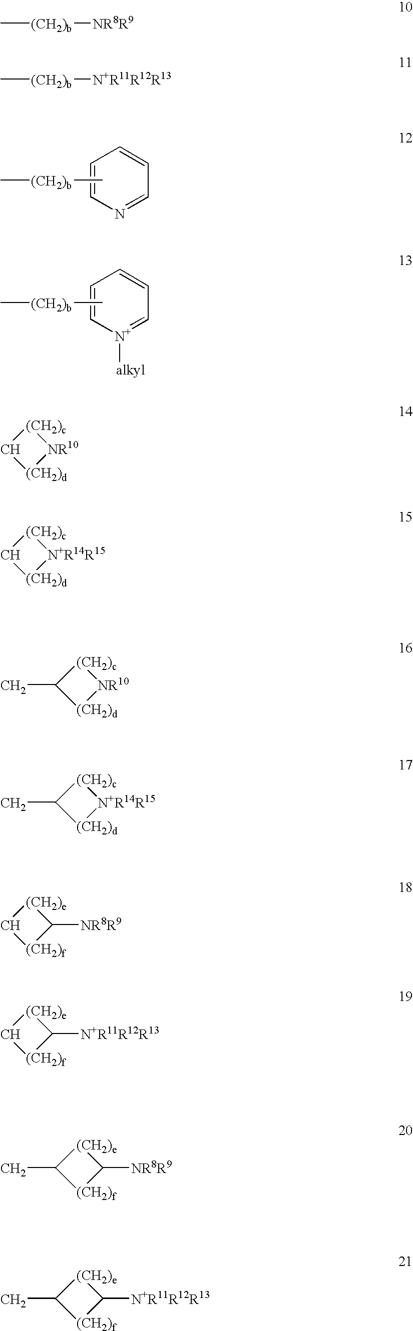 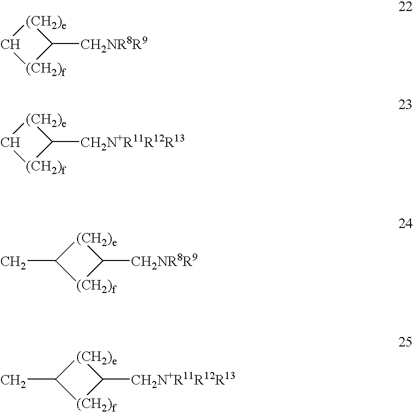
R⁷ is selected from H, alkyl and any group as defined above for R⁶;
R⁸, R⁹ and R¹⁰ are independently selected from H and alkyl, or R⁸ and R⁹ together may be —(CH₂)_{g}-;
R¹¹, R¹², R¹³, R¹⁴ and R¹⁵ are all alkyl, or R¹¹ and R¹² together or R¹⁴ and R¹⁵ together may be —(CH₂)_{g}-;
R¹⁶, R¹⁷ and R¹⁸ are independently selected from H and alkyl, or R¹⁷ and R¹⁸ together may be —(CH₂)ⱼ-;
R¹⁹, R²⁰, R²¹, R²² and R²³ are all alkyl, or R¹⁹ and R²⁰ together or R²¹ and R²² together may be —(CH₂)ⱼ-;
R²⁴ and R²⁵ are independently selected from alkyl, Ar and —(CH₂)ₖ-Ar;
R²⁶ is selected from H, alkyl, optionally substituted phenyl, pyridyl, thienyl, OH, O-alkyl, NH₂, NH-alkyl, N(alkyl)₂, CO₂H, CO₂-alkyl, CONH₂, CONH-alkyl, CON(alkyl)₂, CN and CF₃;
R²⁷ is selected from OH, O-alkyl, O-CO-alkyl, NH₂, NH-alkyl and N(alkyl)₂;
Ar is selected from thienyl and optionally substituted phenyl;
a is 2 or 3, b is 1, 2 or 3; c is 1 or 2, d is 1, 2 or 3; e is 1 or 2; f is 1, 2 or 3; g is 4, 5 or 6; h is 1, 2 or 3; i is 1, 2, 3 or 4; j is 4, 5 or 6; k is 1, 2 or 3; l is 1 or 2; m is 1, 2 or 3; and n is 2, 3 or 4;
provided that:
not more than one of A⁸, A⁹ and A¹⁰ is NH, N(CH₂)ₘR²⁶ or S;
A⁷ and A¹¹ are not both simultaneously N;
neither A⁷ nor A¹¹ is N if one of A⁸, A⁹ and A¹⁰ is NH, N(CH₂)ₘR²⁶ or S;
if A¹⁰ is —CH═CH— then A⁸ is N, A⁹ is CH and both A⁷ and A¹¹ are C;
if A¹⁰ is not —CH═CH— then one of A⁸, A⁹ and A¹⁰ is NH, N(CH₂)ₘR²⁶ or S or one of A⁷ and A¹¹ is N;
not more than one of A¹⁴, A¹⁵ and A¹⁶ is NH, N—CH₃ or S;
A¹² and A¹³ are not both simultaneously N;
if one of A¹⁴, A¹⁵ and A¹⁶ is NH, N—CH₃ or S then A¹² and A¹³ are both C; and
one of A¹⁴, A¹⁵ and A¹⁶ is NH, N—CH₃ or S or one of A¹² and A¹³ is N.
one of A¹⁴, A¹⁵ and A¹⁶ is NH, N—CH₃ or S or one of A¹² and A¹³ is N.
one of A¹⁴, A¹⁵ and A¹⁶ is NH, N—CH₃ or S or one of A¹² and A¹³ is N.

2. A compound according to claim 1 wherein at least one of R¹, R² and R³ is H and one is not H.

3. A compound according to claim 1 wherein one of R¹, R² and R³ is selected from alkyl, F, Cl and Br and the others are H.

4. A compound according to claim 1 wherein R¹ is methyl or Cl and R² and R³ are H.

5. A compound according to claim 1 wherein one of R⁴ and R⁵is H and the other is O-alkyl.

6. A compound according to claim 1 wherein one of R⁴ and R⁵ is H and the other is O-methyl.

7. A compound according to claim 1 wherein R⁴ and R⁵ are both H.

8. A compound according to claim 1 wherein R⁴ and R⁵ are both F.

9. A compound according to claim 1 wherein X¹ is NH.

10. A compound according to claim 1 wherein G¹ is a group according to general formula 2.

11. A compound according to claim 1 wherein G¹ is a group according to general formula 3.

12. A compound according to claim 1 wherein G² is a group according to general formula 4.

13. A compound according to claim 1 wherein G² is a group according to general formula 5, 6, 7, 8 or 9.

14. A compound according to claim 1 wherein G² is a group according to general formula 5, A¹ is CH₂ and A² is NH.

15. A compound according to claim 1 wherein G² is a group according to general formula 5, A¹ is NH or N-alkyl and A² is C(═O).

16. A compound according to claim 1 wherein G² is a group according to general formula 5, 6 or 9, A³is S and A⁴ and A⁵ are both CH.

17. A compound according to claim 1 wherein G² is a group according to general formula 5, 6 or 9, A³ is —CH═CH— and A⁴ and A⁵ are both CH.

18. A compound according to claim 1 wherein R¹ is methyl or Cl, R² and R³ are both H, R⁴ is H or O-methyl, R⁵is H, X¹is NH and Y is S.

19. A compound according to claim 1 wherein R¹ is methyl or Cl, R² and R³ are both H, R⁴ is H or O-methyl, R⁵ is H, X¹ is NH and Y is O.

20. A compound according to claim 1 wherein R² and R³ are both H, X¹is NH and G¹ is

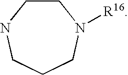

21. A compound according to claim 1 wherein R¹ is methyl or Cl, R² and R³ are both H, R⁴ is H or O-methyl, R⁵ is H, X¹ is NH and G² is

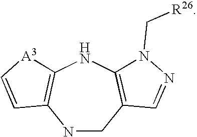

22. A compound according to claim 1 wherein R¹ is methyl or Cl, R² and R³ are both H, R⁴ is H or O-methyl, R⁵ is H, X¹ is NH, G¹ is 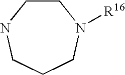
and G¹ is

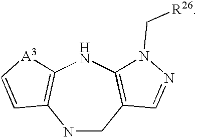

23. A compound according to claim 1 selected from
4-methyl-1-(N-(2-methyl-4-(2,3,4,5-tetrahydro-1,5-benzodiazepin-4-on-1-ylcarbonyl)benzylcarbamoyl)-L-thioprolyl)perhydro-1,4-diazepine,
4-methyl-1-(N-(2-methyl-4-(1-methyl-4,10-dihydropyrazolo[5,4-b][1,5]benzodiazepin-5-yl-carbonyl)benzylcarbamoyl)-L-thioprolyl)perhydro-1,4-diazepine,
4,4-dimethyl-1-(N-(2-methyl-4-(1-methyl-4,10-dihydropyrazolo[5,4-b][1,5]benzodiazepin-5-yl-carbonyl)benzylcarbamoyl)-L-thioprolyl)perhydro-1,4-diazepinium iodide,
4-methyl-1-(N-(2-methyl-4-(5,6,7,8-tetrahydrothieno[3,2-b]azepin-4-ylcarbonyl)benzylcarbamoyl)-L-thioprolyl)perhydro-1,4-diazepine,
4-methyl-1-(N-(2-methyl-4-(5,6,7,8-tetrahydrothieno[3,2-b]azepin-4-ylcarbonyl)benzyloxycarbonyl)-L-prolyl)perhydro-1,4-diazepine,
(4R)-N^{α}-(2-chloro-4-(5,6,7,8-tetrahydrothieno[3,2-b]azepin-4-ylcarbonyl)benzylcarbamoyl)-4-methoxy-L-proline-N-methyl-N-(2-picolyl)amide, and
1-((4R)-N^{α}-(2-chloro-4-(5,6,7,8-tetrahydrothieno[3,2-b]azepin-4-ylcarbonyl)benzylcarbamoyl)-4-methoxy-L-prolyl)-4-(1-pyrrolidinyl)piperidine.

24. A pharmaceutical composition which comprises a compound according to claim 1 as an active agent.

25. A pharmaceutical composition according to claim 24 which is a tablet or capsule for oral administration.

26. A pharmaceutical composition according to claim 24 which is for the treatment of male erectile dysfunction.

27. A use for a compound according to claim 1, which is as a component in the manufacture of a pharmaceutical composition.

28. A use according to claim 27 wherein the pharmaceutical composition is to be used in the treatment of male erectile dysfunction.

29. A method of treating male or female sexual disorders which comprises the administration to a person in need of such treatment of an effective amount of a compound according to claim 1.

30. One or more optical isomers of a compound according to claim 1.
